(19) United States
(12) Patent Application Publication
(10) Pub. No.: US 2023/0210849 A1
(43) Pub. Date: Jul. 6, 2023

(54) CADHERIN-11 INHIBITOR FORMULATION AND ITS USES IN IMMUNOTHERAPY

(71) Applicant: GEORGETOWN UNIVERSITY, Washington, DC (US)

(72) Inventors: Sivanesan DAKSHANAMURTHY, Herndon, VA (US); Ivana PERAN, Washington, DC (US); Stephen W. BYERS, Takoma Park, MD (US); Shahin ASSEFNIA, Washington, DC (US)

(73) Assignee: GEORGETOWN UNIVERSITY, Washington, DC (US)

(21) Appl. No.: 18/000,752

(22) PCT Filed: Jun. 4, 2021
(86) PCT No.: PCT/US21/35950

§ 371 Date: Dec. 5, 2022

Related U.S. Application Data
(60) Provisional application No. 63/035,446, filed on Jun. 5, 2020.

(51) Int. Cl. A61K 31/498; A61K 31/343; A61K 31/473; A61K 31/405; A61K 31/55; A61K 31/4706; A61K 31/423; A61K 31/4196; A61K 31/366; A61P 35/00; A61P 1/18; A61K 31/7068; A61K 31/4418; A61K 31/4045

(52) U.S. Cl. CPC A61K 31/498; A61K 31/343; A61K 31/473; A61K 31/405; A61K 31/55; A61K 31/4706; A61K 31/423; A61K 31/4196; A61K 31/366; A61P 35/00; A61P 1/18; A61K 31/7068; A61K 31/4418; A61K 31/4045

(57) ABSTRACT

Provided herein are compositions and methods for the treatment and prevention of cadherin-11 (CDH11) related diseases. The compositions include CDH11 inhibitors that are formulated for use in combination with chemotherapy and/or immunotherapy, to treat or prevent cancer, fibrosis, and autoimmune diseases.

### PRIOR RELATED APPLICATIONS

This application claims priority to U.S. Provisional Application No. 63/035,446, filed Jun. 5, 2020, which is hereby incorporated in its entirety by this reference.

### STATEMENT REGARDING FEDERALLY FUNDED RESEARCH

This invention was made with government support under Grant No. R01 CA170653 awarded by the National Institutes of Health. The government has certain rights in the invention.

### BACKGROUND

Numerous diseases, for example, cancer and autoimmune disorders, are characterized by the deleterious effects of activated fibroblasts. In cancer, for example, cancer-associated fibroblasts (CAFs) are one of the most abundant and critical components of the tumor mesenchyme, which not only provide physical support for tumor cells but also play a key role in promoting tumorigenesis. Activated fibroblasts are also associated with many immune, fibrotic, and inflammatory responses. However, there is a lack of therapies that effectively target activated fibroblasts to leverage the immune system against disease.

### SUMMARY

Provided herein are compositions and methods for the treatment and prevention of cadherin-11 (CDH11) related diseases, for example, cancer, fibrosis, and autoimmune disorders. The compositions comprise, in a solution: (a) about 5% to about 15% of an organic solvent (w/w); (b) about 20% to about 45% polyethylene glycol (PEG) (w/w); and (c) a compound of the following formula:

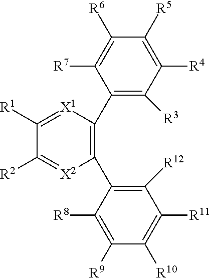

or a pharmaceutically acceptable salt or prodrug thereof, wherein:

R¹, R², R³, R⁴, R⁵, R⁶, R⁷, R⁸, R⁹, R¹⁰, R¹¹, and R¹² are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and

X¹ and X² are each independently selected from CH or N.

In some compositions, the compound has the following formula:

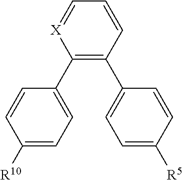

or a pharmaceutically acceptable salt or prodrug thereof, wherein:

R⁵ and R¹⁰ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and

X is selected from CH or N.

In some compositions, the compound is

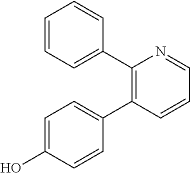 [SMILES: Oc1ccc(-c2cccnc2-c2ccccc2)cc1]

or a pharmaceutically acceptable salt or prodrug thereof.

In some compositions, the compound has the following formula:

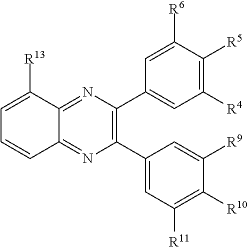

or a pharmaceutically acceptable salt or prodrug thereof, wherein:

R⁴, R⁵, R⁶, R⁹, R¹⁰, R¹¹, and R¹³ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl.

In some compositions, the compound is:

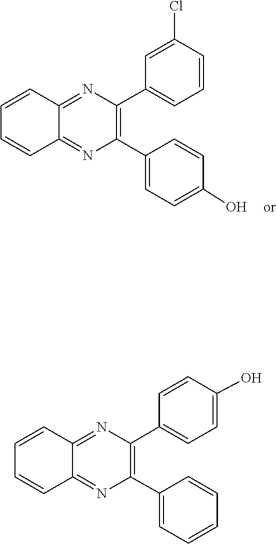 [SMILES: Oc1ccc(-c2nc3ccccc3nc2-c2cccc(Cl)c2)cc1 ; Oc1ccc(-c2nc3ccccc3nc2-c2ccccc2)cc1]

or a pharmaceutically acceptable salt or prodrug thereof

Any of the compositions provided herein, can further comprise a chemotherapeutic agent or an immunotherapeutic agent.

Also provided are methods of treating or preventing a cadherin-11 related disease. The methods comprise administering to a subject in need thereof: (a) an effective amount of any of the compositions described herein and; (b) a chemotherapeutic agent or an immunotherapeutic agent.

### DESCRIPTION OF THE DRAWINGS

FIGS. 1A-I show that CDH11 is significantly increased in human and mouse pancreatic ductal adenocarcinoma (PDAC) CAFs and pancreatitis stroma. (A) CDH11 mRNA in pancreatic carcinoma, ductal (d.) adenocarcinoma, and pancreatitis compared to normal pancreas. Box defines 25th to 75th percentiles, horizontal line defines median, and whiskers minimum and maximum. The fold change (F.C.) is indicated. P-values were determined by unpaired two-tailed t-test. Representative histology of: (B) human normal pancreas and PDAC stained with MTS and CDH11 IHC (5B2H5); (C) human pancreatitis stained with H&E and CDH11 IHC (5B2H5); (D) mouse PanIN and PDAC isolated from KPC mice stained with MTS and CDH11 IHC (5B2H5). (B-D) Black arrows represent CDH11-positive staining in PSCs; asterisks depict CDH11-negative staining in cancer epithelial cells; arrows represent positive MTS staining; scale bars: 50 μm. (E) CDH11 mRNA expression in human and mouse PDAC epithelial cell lines and primary human PSC and CAF cell lines. MDA-MB-231 cell line was used as a positive control for CDH11 expression. (F) Immunoblot for CDH11 (5B2H5) and E-cadherin (E-cad) in PDAC epithelial, PSC and CAF cell lines. (G) Immunofluorescent staining of primary human CAF cell line, 1A1399 for CDH11 (5B2H5) and αSMA. Scale bars: 50 μm. Single cell RNAseq analysis of (H) KPC mouse tumors and (I) immune-cell-depleted s.c. mT3 pancreatic tumors (mT3 cells were derived from a PDAC of a C57BL/6 mouse (Boj et al. Cell 160: 324-338 (2015)). Cell clusters from 10× Genomics scRNAseq analysis visualized by Uniform Manifold Approximation and Projection (UMAP). Feature plots (in the boxes), show CDH11 expression in different cell types from pancreatic cancer microenvironment.

FIGS. 2A-L shows that genetic targeting of CDH11 significantly prolongs survival of PDAC-bearing mice, affects stromal activation and FOXP3+ cell localization. (A) A Kaplan-Meier plot for KPC/CDH11+/+, KPC/CDH11+/− and KPC/CDH11−/− mice. P-values were determined by log-rank Mantel-Cox test. (B) Mass of pancreas at the time of euthanasia. Each data point represents a measurement from an individual mouse. P-values were determined by unpaired two-tailed t-test. (C) αSMA IHC of pancreata. Lines in lower panels outline early PanIN lesions. Scale bars: 100 μm. (D) Quantification of αSMA+ IHC staining represented as % area of pancreata. P-values were determined by unpaired two-tailed t-test. (E) Quantification of tertiary lymphoid structures (TLS) in H&E stained pancreata of KPC mice, by using ImageJ. Each data point represents a ratio of total TLS area over pancreata area from an individual mouse. P-values were determined by unpaired two-tailed t-test. (F) Representative pictures of FOXP3 IHC staining of PDAC tumor center (>300 μm from the tissue margin) from KPC mice. Yellow arrows represent FOXP3-positive staining. Scale bars: 100 μm. Quantification of FOXP3+ cells in the (G) center, (H) periphery, and (I) ratio of FOXP3+ cells in center versus periphery of KPC mice. Minimum five pictures taken at 40× magnification from the center, and the periphery of each tumor were analyzed. Each data point represents an individual PDAC sample. P-values were determined by unpaired two-tailed t-tests. (J) S.C. mPDAC tumor growth in CDH11+/+ and CDH11−/− allografted mice. P-value for interaction was determined by two-way ANOVA. (K) A Kaplan-Meier plot of mPDAC engrafted CDH11+/+ and CDH11−/− mice. P-value was determined by log-rank Mantel-Cox test. (L) S.C. mPDAC tumor growth in the long-term CDH11−/− survivors versus CDH11+/+ mice, at the initial challenge and upon re-challenge with the same mPDAC cell line.

FIGS. 3A-K show that PDAC tissues from KPC/CDH11+/− mice show increase in antigen processing and presentation, and decrease in immunosuppression-associated markers. Differential gene expression analysis of pooled pancreata samples from KPC/CDH11+/− mice (n=5) normalized to KPC/CDH11+/+(n=7). Top 20 genes with (A) increased, and (B) decreased expression; and expression of (C) antigen processing and presentation, (D) cDC, (E) M1 macrophage, (F) M2 macrophage, (G) B cell, and (H) MDSC markers. Cytokine analysis of pooled (I) serum, and (J) pancreata samples of KPC/CDH11+/+(n=3 serum; n=7 pancreata) and KPC/CDH11+/− mice (n=3 serum; n=6 pancreata), performed in duplicates. P-values were determined by unpaired two-tailed t-tests, and reported as *P<0.05, **P<0.01, ***P<0.001. FIGS. 3A-J show that PDAC tissues from KPC/CDH11+/− mice show increase in antigen processing and presentation, and decrease in immunosuppression-associated markers. Differential gene expression analysis of pooled pancreata samples from KPC/CDH11+/− mice (n=5) normalized to KPC/CDH11+/+(n=7). Top 20 genes with (A) increased, and (B) decreased expression; and expression of (C) antigen processing and presentation, (D) cDC, (E) M1 macrophage, (F) M2 macrophage, (G) B cell, and (H) MDSC markers. Cytokine analysis of pooled (I) serum, and (J) pancreata samples of KPC/CDH11+/+(n=3 serum; n=7 pancreata) and KPC/CDH11+/− mice (n=3 serum; n=6 pancreata), performed in duplicates. P-values were determined by unpaired two-tailed t-tests, and reported as *P<0.05, **P<0.01, ***P<0.001. (K) IMC for CD4, CD8a, and CD19 proteins coupled with RNAscope for Ccl21a mRNA performed on PDAC samples from KPC/Cdh11^{+/+} and KPC/Cdh11^{+/-} mice. Bigger panels represent merge staining and smaller panels represent single staining of the same region. DapB was used as a negative control (NC) for mRNA detection by RNAscope. Six regions of interest per PDAC sample (n=3 mice per genotype) were analyzed and quantified by ImageJ using integrated density (IntDen) of the signal. P values were determined by unpaired 2-tailed t test. Arrowheads point to positive staining. Scale bars: 50 mm.

FIGS. 4A-D show that genetic targeting of CDH11 promotes response to gemcitabine. A Kaplan-Meier plot of KPC/CDH11+/+ and KPC/CDH11− (KPC/CDH11+/− and KPC/CDH11−/−) mice treated with: (A) PD1 mAb; or (B) gemcitabine (GEM). Duration, dose and treatment schedule are indicated on the graphs. P-value was determined by log-rank Mantel-Cox test. Cytokine analysis of pooled (C) serum, and (D) pancreata samples of KPC/CDH11+/+(n=3 serum; n=4 pancreata) and KPC/CDH11+/− mice (n=3 serum; n=5 pancreata) treated with gemcitabine, performed in duplicates. P-values were determined by unpaired two-tailed t-test, and reported as *P<0.05, **P<0.01, ***P<0.001, ****P<0.0001.

FIGS. 5A-I show that effective attenuation of pancreatic cancer growth upon CDH11-targeting requires the presence of T and B cells. C57BL/6J mice engrafted with mT3 (derived from a PDAC of a C57BL/6 mouse 23) cells treated with (A) gemcitabine (GEM), (B) CDH11 mAb, (C) CDH11 mAb+ GEM. (D) Binding model of small molecule CDH11-inhibitor, SD133 with the extracellular domain 1 of CDH11. C57BL/6J mice engrafted with mT3 cells treated with (E) SD133 for 2 weeks, (F) SD133 for 2 weeks+ GEM, (G) SD133 for 5 weeks. (H) C57BL/6J mice with pre-existing mT3 tumors treated with SD133 at 40 mg/kg or 10 mg/kg. (I) Immunocompromised Rag1-mutant mice (on C57BL/6J background) with pre-existing mT3 tumors treated with SD133 at 40 mg/kg. The dose, route and treatment schedule are indicated on graphs. P-values for interaction were determined by two-way ANOVA.

FIG. 6 shows a differential CDH11 immunohistochemical-staining pattern using two different antibodies in human tissues. Human normal pancreas, pancreatic ductal adenocarcinoma (PDAC), normal breast and breast cancer specimens were stained with either 5B2H5 or 23C6 clones. Epithelial cells in normal human pancreas are devoid of CDH11 staining using the 5B2H5 clone. However, using the same antibody (23C6) as Birtolo et al. (Am. J Pathol. 187: 146-155 (2018), they stain positive. CDH11 expression is absent in malignant epithelial cells in PDAC patients; however stromal PSCs surrounding the epithelia, stain positively with the 5B2H5 clone (black arrows). On contrary, 23C6 stains positive both the malignant epithelial cells and PSCs in PDAC. Similarly, normal mammary epithelial cells lack CDH11 expression when stained with the 5B2H5 clone (red arrowheads). In contrast, the same type of cells stains positive with the 23C6 clone (black arrowheads). We did show previously that the epithelial component of certain invasive breast cancers and mesenchymal-like breast cancer cell lines do express CDH11 9,10. Indeed, breast cancer epithelial cells stain positive for CDH11 expression after staining with either 5B2H5 or 23C6 antibody Insets are the magnification of the smaller boxes. Original magnification: 40×, insets: 60×. Scale bars: 50 μm.

FIGS. 7A-B show that genetic targeting of CDH11 in an allograft mouse model reduces stromal activation and prevents excess collagen deposition. Representative histology of: (A) s.c., and (B) pancreatic mPDAC tumors in CDH11+/+ and CDH11−/− mice stained for αSMA and MTS, at week 1, 2 and 3 post mPDAC engraftment, and respective quantification of αSMA and MTS staining. P-values were determined by unpaired two-tailed t-test. Each data point represents an individual PDAC sample. P-values were determined by unpaired two-tailed t-test/

FIGS. 8A-B show gene expression in pancreata of KPC/CDH11+/− mice normalized to KPC/CDH11+/+. (A) Validation of Nanostring gene expression analysis by TaqMan probe/primer specific qPCR. The plots represent mean and standard error of the mean of 7 individually ran KPC/CDH11+/+ pancreata samples, and 5 individually ran KPC/CDH11+/− pancreata samples. P-values were determined by unpaired two-tailed t-test, and reported as *P<0.05, **P<0.01, ***P<0.001, ****P<0.0001. (B) Through Immunological Genome Project 11, we queried SKYLINE dataset across 34 immune (related) cell types (http://rstats.immgen.org/Skyline/skyline.html) for top 20 increased (FIG. 3A) and decreased (FIG. 3B) genes in tumors of KPC/CDH11+/− mice over KPC/CDH11+/+. Genes increased in KPC/CDH11+/− tumors are commonly highly expressed in T cells and NK cell, while genes that are decreased in KPC/CDH11+/− tumors are associated with neutrophils, monocytes and macrophages.

FIGS. 9A-C show that CDH11-deficiency impacts signal transduction and cytokine protein expression in PDAC tumors and serum of KPC transgenic mice. (A) PDAC tissues collected at the time of euthanasia from KPC/CDH11+/+ and KPC/CDH11+/− mice were analyzed by phospho protein explorer array (Phospho Explorer Array, Full Moon BioSystems). (B) Serum and (C) PDAC tissues collected at time of euthanasia from KPC/CDH11+/+ and KPC/CDH11+/− mice that were untreated or gemcitabine-treated, were analyzed for cytokine protein expression (Mouse XL Cytokine Array, R&D Systems). Number (n) of samples from individual mice belonging to the same experimental group is indicated in each panel.

FIGS. 10A-B show that genotype has higher impact on gene expression based clustering than gemcitabine treatment. (A) Hierarchical clustering and (B) principal component analysis of differentially expressed genes from PDAC tissues of untreated or gemcitabine-treated KPC transgenic mice, obtained through RNAseq. PDAC tissues collected at the time of euthanasia from KPC/CDH11+/+(wt) and KPC/CDH11+/−(het) mice that were either untreated/control (c) or treated with gemcitabine (g). Each experimental group consisted of 3 mice.

FIGS. 11A-L show that treatment with gemcitabine, CDH11 mAb+/− gemcitabine, or SD133+/− gemcitabine, does not affect mouse body weight. Body weight of C57BL/6J mice engrafted with syngeneic mT3 cells and treated with (A) vehicle (DMSO), (B) gemcitabine, (C) CDH11 mAb, (D) CDH11 mAb+ gemcitabine, (E) SD133 at 150 mg/kg for 2 weeks, (F) SD133 at 150 mg/kg for 2 weeks+ gemcitabine; (G) vehicle (10% DMSO in 30% PEG400), (H) SD133 at 150 mg/kg for 5 weeks, and mice with pre-existing mT3 tumors treated with (I) SD133 at 40 mg/kg, and (J) SD133 at 10 mg/kg. Mouse body weight was measured once or twice a week. Body weight of immunodeficient Rag1-mutant mice on C57BL/6J background with pre-existing syngeneic mT3 tumors treated with (K) vehicle (10% DMSO in 30% PEG400), and (L) SD133 at 40 mg/kg. Gender of mice is indicated in the graphs by commonly used gender symbols. The dose, route and treatment schedule are indicated in colored bars under the x-axes.

FIGS. 12A-B show scRNAseq analysis of KPC mouse tumors (GSE114417). (A) (A) Dot plots showing the expression of selected markers of various cell types. Dot size represents the fraction of cells expressing a specific marker in a particular cluster and intensity indicates the average expression in that cluster. (B) Feature plots showing the expression of commonly used markers in various PDAC cell types. In the box: cell clusters from 10″ Genomics scRNAseq analysis visualized by Uniform Manifold Approximation and Projection (UMAP).

FIGS. 13A-B show scRNAseq analysis of immune-depleted subcutaneous pancreatic tumors from mT3 transplant mouse model. (A) Dot plots showing the expression of selected markers of various cell types. Dot size represents the fraction of cells expressing a specific marker in a particular cluster and intensity indicates the average expression in that cluster. (B) Feature plots showing the expression of commonly used markers in various cell types from pancreatic cancer microenvironment. In the box: cell clusters from 10× Genomics scRNAseq analysis visualized by Uniform Manifold Approximation and Projection (UMAP).

FIGS. 14A-C show CDH11 expression in KPC/Cdh11 [symbol] / [symbol] and KPC/Cdh11−/− mouse pancreatic tissue lysates. (A)Cdh11 mRNA expression in mouse pancreatic tissue lysates. (B) Immunoblot for CDH11 (5B2H5) in mouse pancreatic tissue lysates. Human primary CAF cell line, 1A1340 was used as a positive control for CDH11 expression. (C) CDH11 IHC (5B2H5) staining of KPC/Cdh11 mouse pancreatic tissues. NC, negative control. Scale bars: 100 mm.

### DETAILED DESCRIPTION

The present disclosure provides compositions comprising cadherin-11 inhibitors that function as immunomodulators. More specifically, the compositions provided herein are effective in decreasing immunosuppression and fibrosis. In some examples, the immunosuppressive state and/or fibrosis are associated with a disease, for example, cancer. In cancer, administration of any of the compositions described herein, effectively targets cadherin-11 on activated fibroblasts that are present in an immunosuppressive tumor environment. Targeting cadherin-11 on activated fibroblasts increases the number of antigen presenting cells, thus promoting an anti-tumor immune environment.

Pancreatic cancer is an exemplary disease that can be treated using the compositions and methods described herein. Pancreatic cancer is soon to be the second leading cause of cancer-related death with an overall median survival of 8-11 months. Even though the mutation events that drive progression of pancreatic ductal adenocarcinoma (PDAC) are well known, it has a 5-year survival rate of about 10%. For 70% of patients, systemic chemotherapy is the only option and this mainly relieves the symptoms and/or slightly extends survival, rather than cures the patients. FDA-approved immunotherapy (i.e. anti-PD1) for microsatellite instability-high solid cancers, is effective in only 1% of pancreatic cancer patients that have defects in mismatch repair genes.

One of the hallmarks of PDAC is extensive desmoplasia/fibrosis that comprises up to 80% of the tumor. Cancer-associated fibroblasts (CAFs), including activated pancreatic stellate cells (PSCs), and their deposition of extracellular matrix influences tumor progression, metastasis, therapy resistance and formation of new blood vessels. CAFs release growth factors and cytokines that not only influence growth of pancreatic cancer cells, but also affect infiltration of immune cells into the tumor microenvironment (TME). Indeed, a third of human PDAC specimens have immune infiltration similar to melanoma, but mainly located in the stromal compartment. While the percentage of CD8⁺ cells in patients varies between <7% and 15-30% of CD45⁺ cells, PDAC is characterized by high numbers of immunosuppressive cells, such as FOXP3⁺ T regulatory cells (T-regs), myeloid-derived suppressive cells (MDSCs) and macrophages. However, depletion of immunosuppressive FOXP3⁺ cells promotes PDAC progression, and depletion of CAFs themselves is just as likely to promote PDAC as inhibit it. As shown herein, it is better to modulate CAF/immune infiltrate cross-talk to limit tumor growth, rather than entirely deplete CAFs from the TME.

### Compositions

Provided herein are compositions comprising, in a solution: (a) about 5% to about 15% of an organic solvent (w/w); (b) about 20% to about 45% polyethylene glycol (PEG) (w/w); and (c) a compound of the following formula:

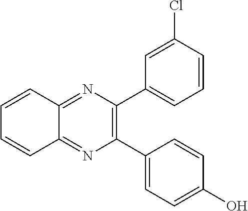 [SMILES: Oc1ccc(-c2nc3ccccc3nc2-c2cccc(Cl)c2)cc1]

or a pharmaceutically acceptable salt or prodrug thereof, wherein:

R¹, R², R³, R⁴, R⁵, R⁶, R⁷, R⁸, R⁹, R¹⁰, R¹¹, and R¹² are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and

X¹ and X² are each independently selected from CH or N.

In some compositions, the compound has the following formula:

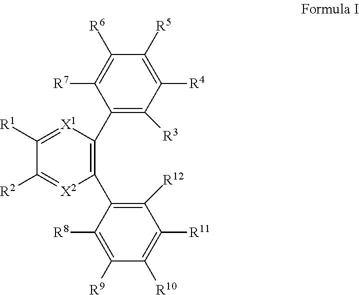

or a pharmaceutically acceptable salt or prodrug thereof, wherein:

R⁵ and R¹⁰ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and

X is selected from CH or N.

In some compositions, the compound is

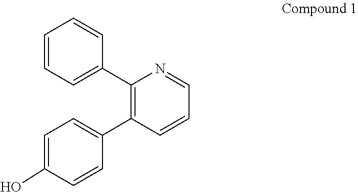 [SMILES: Oc1ccc(-c2cccnc2-c2ccccc2)cc1]

or a pharmaceutically acceptable salt or prodrug thereof. Compound I is also known as SD133.

In some compositions, the compound has the following formula:

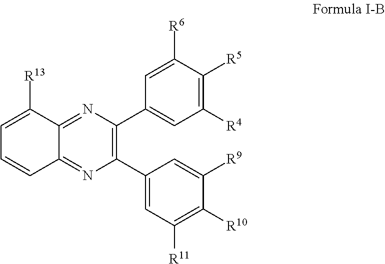

or a pharmaceutically acceptable salt or prodrug thereof, wherein:

R⁴, R⁵, R⁶, R⁹, R¹⁰, R¹¹, and R¹³ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl.

Examples of Formula I-B include, but are not limited to, the following compounds:

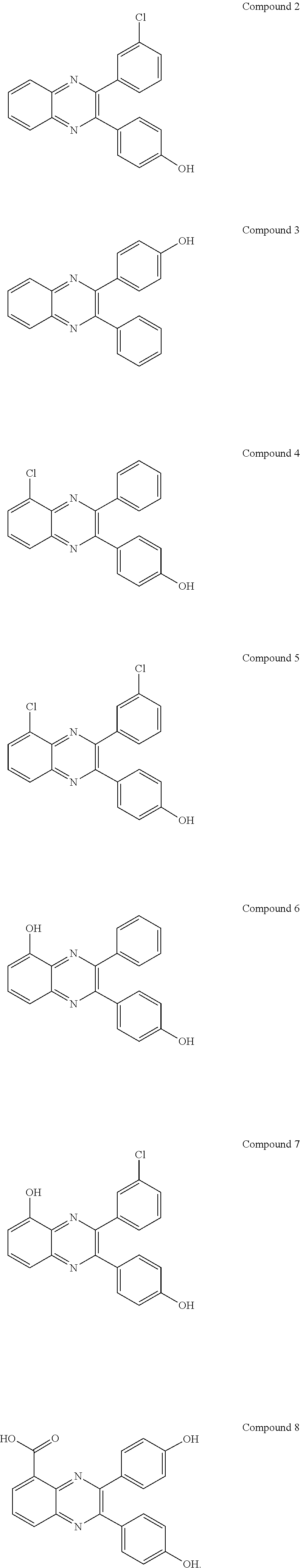

In some compositions, the compound is:

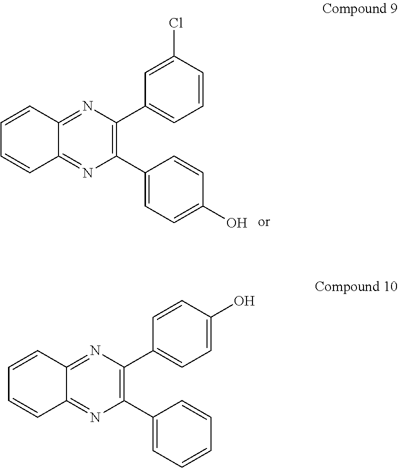 [SMILES: Oc1ccc(-c2nc3ccccc3nc2-c2cccc(Cl)c2)cc1 ; "or" ; Oc1ccc(-c2nc3ccccc3nc2-c2ccccc2)cc1]

or a pharmaceutically acceptable salt or prodrug thereof

Another class of cadherin-11 inhibitors that can be used in the compositions and methods provided herein is represented by Formula II:

A class of cadherin-11 inhibitors useful in the methods described herein is represented by Formula II:

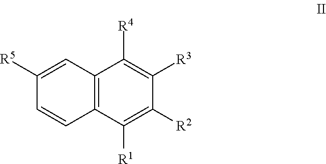

or a pharmaceutically acceptable salt or prodrug thereof.

In Formula II, R¹, R², R³, R⁴, and R⁵ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl.

In Formula II, adjacent R groups on the phenyl ring, i.e., R¹, R², R³, and R⁴, can be combined to form substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl groups. For example, R¹ can be a ethylene group and R² can be an methanimine group that combine to form a C₆ heteroaryl. Other adjacent R groups include the combinations of R² and R³, and R³ and R⁴.

Examples of Formula II include, but are not limited to:

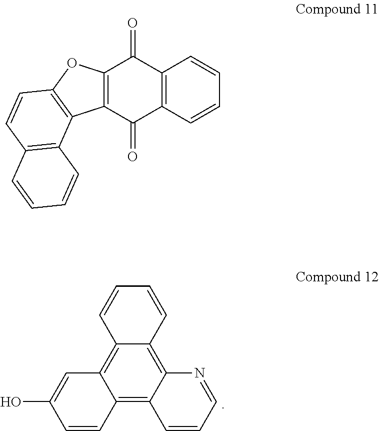 [SMILES: O=C1c2ccccc2C(=O)c2c1oc1ccc3ccccc3c21 ; Oc1ccc2c(c1)c1ccccc1c1ncccc21]

Compounds represented by Formula I or Formula II are cadherin-11 inhibitors. Additional cadherin-11 inhibitors useful in the compositions and methods described herein have also been identified that may not be represented by Formula I or Formula II. The structures of these cadherin-11 inhibitors are as follows:

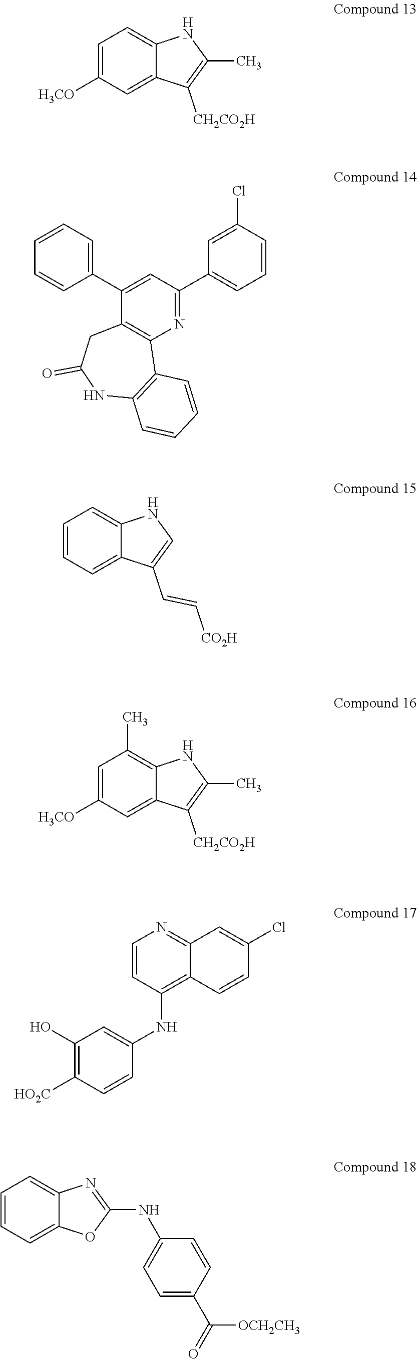

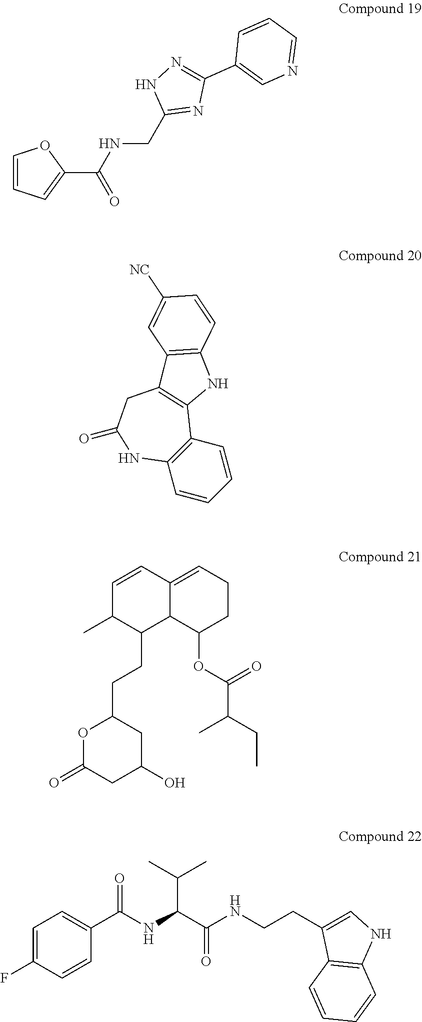 [SMILES: O=C(NCc1nc(-c2cccnc2)n[nH]1)c1ccco1 ; N#Cc1ccc2[nH]c3c(c2c1)CC(=O)Nc1ccccc1-3 ; CCC(C)C(=O)OC1CCC=C2C=CC(C)C(CCC3CC(O)CC(=O)O3)C21 ; CC(C)[C@H](NC(=O)c1ccc(F)cc1)C(=O)NCCc1c[nH]c2ccccc12]

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline.

The alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl molecules used herein can be substituted or unsubstituted. As used herein, the term substituted includes the addition of an alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl group to a position attached to the main chain of the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term unsubstituted indicates the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl has a full complement of hydrogens, i.e., commensurate with its saturation level, with no substitutions, e.g., linear decane (—(CH₂)₉—CH₃).

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I, Formula II, and the additional cadherin-11 inhibitors described above include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography. Additional information regarding synthesis of the compounds provided herein can be found in U.S. Pat. No. 8,802,687, incorporated in its entirety by this reference.

Any of the compositions or formulations described herein can be made or formulated in a solution by dissolving any of the compounds described herein in an organic solvent and diluting the solution with polyethylene glycol, such that the final concentration (w/w) of the organic solvent is between about 5% to about 15% and the concentration of the polyethylene glycol (w/w) is between about 20% to about 45%.

In some compositions, the concentration of the organic solvent (w/w) is about 5, 6, 7, 8, 9, 10, 11, 12 13, 14, or 15% (w/w) or any percentage in between these percentages. In some compositions, the concentration of the polyethylene glycol (w/w) is about 20, 21, 22, 23, 24, 25, 26, 27, 28, 29, 30, 31, 32, 33, 34, 35, 36, 37, 38, 39, 40, 41, 42, 43, 44, 45% (w/w) or any percentage in between these percentages. In some compositions, the concentration of water (w/w) is about 75%, 80%, 85%, 90%, 95% or any other percentage in between these percentages. In some compositions, the concentration of organic solvent is about 10% (w/w) and the concentration of the polyethylene glycol is about 30% (w/w).

In some compositions, the organic solvent is selected from the group consisting of acetone, ethyl acetate, hexane, heptane, dichloromethane, methanol, ethanol, tetrahydrofuran, acetonitrile, dimethylformamide, toluene, dimethylsulfoxide (DMSO). In some compositions, the polyethylene glycol is selected from the group consisting of PEG-400, PEG-100, PEG-200, PEG-300, PEG-600, PEG-800, PEG-1000, PEG-2000, PEG-3000, PEG-4000, PEG-5000, PEG-6000, PEG-7000, PEG-8000, PEG-9000, and PEG-10,000. In some examples, the composition comprises two or more polyethylene glycols selected from the group consisting of PEG-400, PEG-100, PEG-200, PEG-300, PEG-600, PEG-800, PEG-1000, PEG-2000, PEG-3000, PEG-4000, PEG-5000, PEG-6000, PEG-7000, PEG-8000, PEG-9000, and PEG-10,000.

In some compositions or formulations, the concentration of DMSO is about 10% (w/w) and the concentration of PEG-400 is about 30% (w/w).

Any of the compositions comprising one or more cadherin-11 inhibitors described herein can further comprise a chemotherapeutic agent and/or an immunotherapeutic agent. Chemotherapeutic agents suitable for co-administration with compositions of the present disclosure include, but are not limited to, for example, taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, gemcitabine, 6-mercaptopurine, 6-thioguanine, cytarabine, 5-fluorouracil decarbazine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, irinotecan, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide.

Immunotherapeutic agents suitable for co-administration with compositions of the present disclosure include, but are not limited to, for example, checkpoint inhibitors (e.g., PD-1 inhibitor, PD-L1 inhibitor, PD-L2 inhibitor, BTLA inhibitor, CTLA4 inhibitor or LAG3 inhibitor) monoclonal antibodies that bind cancer antigens; immune system modulators (e.g., cytokines (for example, interferons, interleukin 12, erythropoietin, interleukin 11, granulocyte-macrophage colony stimulating factor, and granulocyte colony stimulation factor, thalidomide, lenalidomide, pomalidomide and imiquimod); or a cancer vaccine.

### Methods

Provided herein is a method for treating or preventing a cadherin-II related disease in a subject comprising administering to the subject in need thereof an effective amount of: (a) any of the compositions described herein; and (b) a chemotherapeutic agent or an immunotherapeutic agent.

In some methods, the cadherin-11 related disease is cancer. As used herein, cancer is a disease characterized by the rapid and uncontrolled growth of aberrant cells. Cancer cells can spread locally or through the bloodstream and lymphatic system to other parts of the body. In some embodiments, the cancer is a solid tumor. In some embodiments, the cancer is a blood or hematological cancer. In some methods, the cancer is selected from the group consisting of pancreatic cancer, lung cancer, colorectal cancer, cervical cancer, skin cancer, renal cancer, liver cancer, breast cancer (for example, estrogen receptor negative or triple negative breast cancer), ovarian cancer, prostate cancer (for example, androgen receptor negative prostate cancer), bladder cancer, gastric cancer, and glioblastoma (for example, mesenchymal subtype glioblastoma).

In some methods, the cadherin-11 related disease is an autoimmune disease. As used herein, an autoimmune disease is a disease where the immune system cannot differentiate between a subject's own cells and foreign cells, thus causing the immune system to mistakenly attack healthy cells in the body. In some methods, the autoimmune disease is selected from the group consisting of inflammatory bowel disease, rheumatoid arthritis, diabetes, metabolic disorder, Sjögrens syndrome, and scleroderma. In some methods, the cadherin-11 related disease is fibrosis and the methods provided herein decrease fibrosis in one or more organs of the subject, for example, intestinal fibrosis, lung fibrosis, heart fibrosis, liver fibrosis or kidney fibrosis. In some methods, fibrosis is associated with cancer or an autoimmune disorder.

In some methods, the cadherin-11 related disease is refractory or resistant to the chemotherapeutic agent or the immunotherapeutic agent, at least in the absence of treatment with any of the compositions comprising a cadherin-11 inhibitor described herein. In some methods, administration of the composition enhances the efficacy of the chemotherapeutic agent or the immunotherapeutic agent. In some methods, composition comprising a cadherin-11 inhibitor and the chemotherapeutic agent or the immunotherapeutic agent act synergistically. In some methods, the amount of the chemotherapeutic agent or immunotherapeutic agent can be deceased, as compared to the dosage of the chemotherapeutic or immunotherapeutic in the absence of treatment with a composition comprising a cadherin-11 inhibitor.

In some methods, the composition comprising a cadherin-11 inhibitor decreases fibrosis in the subject, for example, fibrosis associated with cancer or an autoimmune disorder. In some methods, the composition comprising a cadherin-11 inhibitor decreases fibrosis associated with lung cancer or pancreatic cancer. Examples of chemotherapeutic agents are described above. In some methods, the chemotherapeutic agent is gemcitabine. In some methods, the immunotherapeutic agent is a checkpoint inhibitor. In some methods, the checkpoint inhibitor is selected from the group consisting of a PD-1 inhibitor, a PD-L1 inhibitors, a PD-L2 inhibitor, a BTLA inhibitor, a CTLA4 inhibitor and a LAG3 inhibitor. In some methods, the checkpoint inhibitor is an antibody that inhibits PD-1, PD-L1, PD-L2, BTLA, CTLA4 or LAG3.

In certain methods, the CTLA4 inhibitor is anti-CTLA-4 antibody, for example, ipilimumab; the PD-1 inhibitor is an anti-PD-1 antibody selected from nivolumab, pembrolizumab, and pidilizumab; and the PD-L1 inhibitor is an anti-PD-L1 antibody selected from (MDX-1105) BMS-936559, MPDL3280A (atezolizumab), MEDI4736 (durvalumab), and MSB0010718C.

It is understood that any of the cadherin 11 inhibitors described herein can be replaced with an anti-CDH11 antibody, for example, a monoclonal anti-CDH11 antibody, as described in the Examples. A cadherin-11 related disease, for example, cancer or an autoimmune disorder, can be treated or prevented by administering an anti-CDH11 antibody and a chemotherapeutic or immunotherapeutic agent to the subject. In some embodiments, an anti-CDH11 antibody, for example, a monoclonal antibody, and gemcitabine are administered to the subject. In some embodiments, the subject has cancer, for example, pancreatic cancer.

Any of the methods provided herein can further comprise diagnosing a subject with a cadherin-II related disease. Any of the methods provided herein can further comprise radiation therapy, immunotherapy, chemotherapy or surgery, cell transplantation therapy (for example, T cell therapy (Hinrichs and Rosenberg “Exploiting the curative potential of adoptive T-cell therapy for cancer” Immunol. Rev. 257(1): 56-71 (2014); Feins et al. “An Introduction to Chimeric Antigen Receptor (CAR) T-cell Immunotherapy for Human Cancer,” Am. J Hematol. 94(S1): S3-S9 (2019)), to name a few.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects. In any of the methods provided herein, the subject can be a subject diagnosed with a cadherin-II disease.

As used herein the terms treatment, treat, or treating refers to a method of reducing one or more of the effects of the disorder or one or more symptoms of the disorder, for example, cancer in the subject. Thus in the disclosed methods, treatment can refer to a 10%, 20%, 30%, 40%, 50%, 60%, 70%, 80%, 90%, or 100% reduction in the severity of cancer. For example, a method for treating cancer is considered to be a treatment if there is a 10% reduction in one or more symptoms of the cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As utilized herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of cancer or one or more symptoms of cancer in a subject susceptible to cancer as compared to control subjects susceptible to cancer that did not receive a composition described herein The disclosed method is also considered to be a prevention if there is a reduction or delay in onset, incidence, severity, or recurrence of cancer or one or more symptoms of cancer in a subject susceptible to cancer after receiving a composition described herein as compared to the subject's progression prior to receiving treatment. Thus, the reduction or delay in onset, incidence, severity, or recurrence of cancer can be about a 10, 20, 30, 40, 50, 60, 70, 80, 90, 100%, or any amount of reduction in between.

As used herein, administer or administration refers to the act of introducing, injecting or otherwise physically delivering a substance as it exists outside the body (e.g., a multi-epitope polypeptide and/or a metabolic inhibitor) into a subject, such as by mucosal, intradermal, intravenous, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The compositions are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539. The composition can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Effective doses for any of the administration methods described herein can be extrapolated from dose-response curves derived from in vitro or animal model test systems.

As used herein, the term therapeutically effective amount or effective amount refers to an amount of a compound, composition, chemotherapeutic agent or immunotherapeutic agent described herein, that, when administered to a subject, is effective to treat a disease or disorder either by one dose or over the course of multiple doses. A suitable dose can depend on a variety of factors including the particular polypeptide used and whether it is used concomitantly with other therapeutic agents. Other factors affecting the dose administered to the subject include, e.g., the type or severity of the disease. For example, a subject having pancreatic cancer may require administration of a different dosage of a composition comprising a cadherin-11 inhibitor and/or a chemotherapeutic agent than a subject with breast cancer.

The effective amount of the compounds described herein or pharmaceutically acceptable salts or prodrugs thereof can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

A pharmaceutical composition can include a therapeutically effective amount of any compound, chemotherapeutic agent, and/or immunotherapeutic agent described herein. In some embodiments, the pharmaceutical composition can further comprise a carrier. Such effective amounts can be readily determined by one of ordinary skill in the art as described above. Considerations include the effect of the administered compound, i.e., cadherin-11 inhibitor, or the combinatorial effect of the compound with one or more additional active agents (for example, a chemotherapeutic agent or immunotherapeutic agent), if more than one agent is used in or with the pharmaceutical composition. Cell culture assays and animal studies can be used in formulating a range of dosage for use in humans.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN© (ICI, Inc.; Bridgewater, N.J.), polyethylene glycol (PEG), and PLURONICS' (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

The agents described herein can be incorporated into pharmaceutical compositions which allow for immediate release or delivery of those agents to a mammal. The agents described herein can also be incorporated into pharmaceutical compositions which allow for modified release, for example, delayed release or extended release (for example, sustained release or controlled release) of those agents to a mammal for a period of several days, several weeks, or a month or more. Such formulations are described, for example, in U.S. Pat. Nos. 5,968,895 and 6,180,608 and are otherwise known in the art. Any pharmaceutically-acceptable, delayed release or sustained-release formulation known in the art is contemplated.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

The term pharmaceutically acceptable salts as used herein refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See Stahl and Wermuth, Pharmaceutical Salts: Properties, Selection, and Use, Wiley-VCH, 2008, which is incorporated herein by reference in its entirety, at least, for compositions taught herein.)

Besides such inert diluents, the composition can also include additional agents, such as wetting, emulsifying, suspending, sweetening, flavoring, or perfuming agents. It is understood that combinations, for example, a cadherin-11 inhibitor or a composition comprising a cadherin-11 inhibitor and a chemotherapeutic agent, are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Human Primary CAF Isolation

Collection of pancreatic tumor samples from patients undergoing Whipple surgery was approved by the Institutional Review Board of Georgetown University, and written informed consent was received from participants prior to inclusion in the study. The diagnosis was established by a pathologist based on histology examination. Specimens underwent collagenase digestion for 15 minutes at 37° C. on a rotor, followed by centrifugation at 600 g for 3 minutes. Supernatant was transferred to a new tube and equal volume of 1×PBS+10% FBS was added. Sample was centrifuged for 3 minutes at 1,500 g, supernatant was discarded and cell pellet was resuspended in 5 mL of 1×PBS+10% FBS, three times. Finally, cell pellet was resuspended in 4 mL of complete Stellate Cell Medium (SteCM, Cat #5301, ScienCell Research Laboratories (Carlsbad, Calif.)), and plated into one well of a 6-well plate, and propagated.

### Mice

The experimental protocols were approved by the Georgetown University Animal Care and Use Committee. A pair of CDH11^{-/-} mice (Horikawa et al. Dev. Biol. 215: 182-189 (1999)) was kindly provided by Dr. Michael Brenner from Harvard University. A breeding pair of p48-Cre;LSL-Kras^{G12D/+};LSL-Trp53^{R172H/+} (KPC) mice (Hingorani et al. Cancer Cell 7: 469-483 (2005)) was provided by Dr. Anton Wellstein from Georgetown University. C57BL/6J mice (Cat #000664; Research Resource Identifier (RRID): IMSR_JAX:000664) and Rag1-mutant mice on C57BL/6J background (Cat #002216; RRID: IMSR_JAX:002216) were purchased from The Jackson Laboratory (Bar Harbor, Me.). In transplant experiments, 12-week old C57BL/6J and Rag1-mutant (on C57BL/6J background) mice were bilaterally subcutaneously (s.c.) injected with 5×10⁵ mT3 pancreatic cancer cells, derived from a Kras^{+/LSL-G12D};Trp53^{+/LSL-R172H};Pdx-Cre mouse on C57BL/6 background. Both genders of mice were equally represented throughout all in vivo experiments. Mice were housed in specific pathogen free environment under standard conditions. See, Bandrowski et al. “The Resource Identification Initiative: A cultural shift in publishing,” F1000Res 4: 134 (2015), regarding Research Resource Identifiers (RRIDs),

### Animal Treatment

Anti-PD1 (InVivoPlus anti-mouse PD1 (CD279), clone RMP1-14, Cat #BP0146, Bio X Cell (Lebanon, N.H.)) was administered intraperitoneally (i.p.) at 250 μg/mouse, 2× week. Gemcitabine (G-4177, LC Laboratories (Woburn, Mass.)) was administered i.p. at 100 mg/kg, 2× week. Treatment with CDH11 monoclonal antibody (mAb), SYN0012 (Roche (Basel, Switzerland)) was administered i.p. at 10 mg/kg, 2× week. Treatment with a small molecule CDH11-inhibitor, SD133, was administered i.p. at 150 mg/kg, 40 mg/kg or 10 mg/kg, 4× week. Stock solution of SD133 at 375 mg/mL was made in DMSO (Sigma (St. Louis, Mo.)), and further diluted in 30% PEG400 (Sigma).

### Cell Culture

Mouse pancreatic cancer cells mPDAC and mT3 cell line were isolated from the pancreatic cancer of a KPC mouse. All mouse (mPDAC, mT3 and Panc02 (RRID: CVCL_D627)) and human pancreatic cancer cells (COL0357 (RRID: CVCL_0221) and Panc-1—available through ATCC, Cat #CRL-1469; RRID: CVCL_0480), as well as human breast cancer cell line (MDA-MB-231—available through ATCC, Cat #HTB-26; RRID: CVCL_0062) were grown in DMEM (Cat #11995-065, Gibco, Invitrogen (Carlsbad, Calif.)) with 10% fetal bovine serum (FBS) without antibiotics. Human pancreatic stellate cells (PSC), HPaSteC, were purchased from ScienCell (Cat #3830, ScienCell Research Laboratories). HPaSteC cells and other primary PSCs were grown in complete Stellate Cell Medium, which includes stellate cell growth supplements and 2% FBS, but without antibiotics (SteCM, Cat #5301, ScienCell Research Laboratories). All cells were grown under regular cell culture growth conditions, and tested negative for mycoplasma.

Mouse Primary Pancreatic Ductal Adenocarcinoma (mPDAC) Cell Isolation

Under sterile conditions, a piece of pancreas with the macroscopically apparent cancer mass was excised and transferred into DMEM/F12 media (Invitrogen, Life Technologies) with addition of penicillin, streptavidin and gentamicin. Tumor tissue was minced and transferred into collagenase solution (35 mL of DMEM/F12+ antibiotics and gentamicin, 70 mg of collagenase, 140 mg of trypsin powder, 1.75 mL of FBS). After incubation at 37° C. for 1.5 hours on a shaker at 150 rpm, tissue was spun at 600 rpm for 10 min. Supernatant was aspirated carefully, and was added to 15 mL of fresh DMEM/F12 media+ antibiotics. Tissue was briefly spun 4 times at 600 rpm, supernatant was aspirated and 10 mL of fresh DMEM/F12 media+ antibiotics was added. After the final spin, primary cells were plated on a collagen coated 10 cm cell culture dish in primary cell media (500 mL of F12 media (Invitrogen, Life Technologies) with antibiotics and gentamicin, 50 mL of FBS, 8 mg of insulin, 5 μg of EGF, 200 μg of hydrocortisone, 2 μg of cholera toxin). Approximately 30 min later, the supernatant containing mPDAC cells was transferred from the cell culture dish to a 6-well plate and primary cells were incubated and propagated under regular cell culture growth conditions. When cells reached confluency, they were plated in a 96-well plate at the density of 1 cell per well, and followed over the next 3-4 weeks until clonal lines (C5, C8, D10, F2, G8, G9) emerged.

### Antibodies

Primary antibodies: CDH11 (5B2H5) monoclonal antibody (Cat #32-1700, Thermo Fisher Scientific (Waltham, Mass.); RRID: AB_2533068), αSMA monoclonal antibody (Cat #ab124964, Abcam (Cambridge, UK); RRID: AB_11129103), E-cadherin monoclonal antibody (Cat #610182, BD Transduction Laboratories (San Jose, Calif.); RRID: AB_397581), CD8 monoclonal antibody (Cat #14-0808, eBioscience; RRID: AB_2572860), CD4 monoclonal antibody (Cat #14-9766, eBioscience (San Diego, Calif.); RRID: AB_2573007), FOXP3 monoclonal antibody (Cat #14-5773, eBioscience; RRID: AB_467575) and GAPDH monoclonal antibody (Cat #10R-G109A, Fitzgerald (Tompkinsville, Ky.); RRID:AB_1285808).

Secondary antibodies: HRP-conjugated anti-mouse IgG (Cat #ab205719, Abcam; RRID: AB_2755049), HRP-conjugated anti-rabbit IgG (Cat #7074, Cell Signaling Technology (Danvers, Mass.); RRID: AB_2099233), anti-mouse IgG Alexa Fluor 488 (Cat #A28175, Thermo Fisher Scientific; RRID: AB_2536161), and anti-rabbit IgG Alexa Fluor 568 (Cat #A-11011, Thermo Fisher Scientific; RRID: AB_143157).

### Immunohistochemistry

Five-micron sections from formalin fixed paraffin embedded tissues were de-paraffinized with xylenes and rehydrated through a graded alcohol series. Heat induced epitope retrieval (HIER) was performed, followed by immunohistochemical staining. Slides were counterstained with hematoxylin (Fisher, Harris Modified Hematoxylin). Consecutive sections with primary antibody omitted were used as negative controls.

### Histologic Quantification

A minimum of five high-powered fields of each tumor sample were captured by Keyence BZ-X700 microscope. The number of positive CD8, CD4 and FOXP3 cells was quantified using ImageJ (https://imagej.nih.gov/ij/; RRID: SCR_003070).

### Immunofluorescence

Cells were grown in a monolayer on a glass coverslip, fixed with ice-cold methanol for 15 minutes, followed by standard immunofluorescence techniques. Negative control substituted antibody for diluent alone during primary antibody incubation step.

Protein Isolation from Cell Lines and Tumor Tissues

Cells were lysed on the plate, or tumor tissues were homogenized in RIPA buffer (Cat #786-490, G-Biosciences (Darmstadt, Germany) with added protease (complete ULTRA Tablets, Mini, EDTA-free, EASYpack, Cat #05892791001, Roche) and phosphatase (PhosSTOP, EASYpack, Cat #04906845001, Roche) inhibitors. Protein concentration was assessed using DC Protein Assay (Cat #5000112, BioRad (Hercules, Calif.)) with BSA standard.

### Western Blotting

Standard immunoblotting techniques were used. Changes in the indicated protein targets were compared to the loading control GAPDH.

### Cytokine Protein Expression

Equal amount of protein lysates or serum belonging to the same experimental group were pooled together and used to run a proteome profiler array by following a protocol provided with the Mouse XL Cytokine Array Kit (Cat #ARY028, R&D Systems (Minneapolis, Minn.)). After developing the image, cytokine expression was quantified by using ImageJ (https://imagej.nih.gov/ij/; RRID: SCR_003070).

### Phospho Protein Expression

Equal amount of protein lysates belonging to the same experimental group were pooled together and used to run the ELISA-based antibody array by following a protocol provided with the Phospho Explorer Array (Cat #PEX100, Full Moon BioSystems (Sunnyvale, Calif.)). Image was scanned and analyzed by Full Moon BioSystems.

RNA Isolation, cDNA Synthesis and Quantitative PCR

RNA was extracted using RNeasy Mini Kit (Cat #74106, Qiagen (Hilden, Germany)). cDNA was synthesized using TaqMan Reverse Transcription Reagents (Cat #N8080234, Invitrogen). All targets were amplified for 40 cycles using gene specific TaqMan primer/probe sets (Applied Biosystems (Foster City, Calif.): human CDH11 Hs00901475_m1, mouse CDH11 Mm00515466_m1, eukaryotic 18S rRNA Hs99999901_s1, mouse Ccl2 Mm00441242_m1, mouse Ccl5 Mm01302427_m1, mouse Ccr7 Mm99999130_s1, mouse Cd3e Mm01179194_m1, mouse Cd207 Mm00523545_m1, mouse Cfd Mm01143935_g1, mouse Chil3 Mm03052453_g1, mouse Glycam1 Mm00801716_m1, mouse H2-Eb1 Mm00439221_m1, mouse Il1r2 Mm00439629_m1, mouse Lgals3 Mm00802901_m1, mouse Tap1 Mm00443188_m1) and TaqMan Fast Universal PCR Master Mix (2×), no AmpErase UNG (Cat #4352042, Applied Biosystems) on a StepOnePlus Real-Time PCR System (Applied Biosystems). Relative gene expression quantification was performed using ΔΔCt method (2^{-ΔΔCt}), and expression levels were normalized to 18S rRNA.

### Nanostring Gene Expression Analysis

Total RNA was extracted from snap frozen pancreatic cancer tissues using RNeasy Mini Kit (Cat #74106, Qiagen). RNA concentration and purity was measured by NanoDrop (Thermo Scientific). RNA samples of equal concentration that belonged to the same experimental group were pooled together and analyzed for RNA integrity by Bioanalyzer 2100 (Agilent Technologies (Santa Clara, Calif.)). Gene expression of 770 genes was performed using the PanCancer Immune Profiling Panel on nCounter system (Cat #XT-CSO-MIP1-12, NanoString Technologies, Inc., Seattle, Wash.). Gene expression data was analyzed by nSolver Analysis Software (NanoString Technologies; http://www.nanostring.com/products/nSolver; RRID: SCR_003420).

### RNA Sequencing (RNAseq)

Total RNA was extracted from snap frozen pancreatic cancer tissues using Direct-zol RNA Miniprep Kit (Cat #R2053, Zymo Research (Irvine, Calif.)). RNA concentration and purity was measured by NanoDrop (Thermo Scientific), and integrity and quality by Bioanalyzer 2100 (Agilent Technologies) and Qubit (Thermo Fisher Scientific). Indexed, single-index sequencing libraries were prepared from total RNA using the Illumina TruSeq Stranded Total RNA Library Preparation Kit (human/mouse/rat) with rRNA depletion and random-primed cDNA synthesis (Cat #20020596, Illumina (San Diego, Calif.)). RNAseq was performed at the Georgetown Lombardi Comprehensive Cancer Center using the Illumina HiSeq 4000 instrument with 150 bp paired end read lengths and 50M raw reads per sample. Reads were trimmed using Fqtrim 0.9.5 and mapped to the GRCm38 build of the Mus Musculus genome using RSEM (http://deweylab.biostat.wisc.edu/rsem/; RRID: SCR_013027). Differential expression for the assembled gene transcripts was done using the R Bioconductor package edgeR (http://bioconductor.org/packages/edgeR/; RRID: SCR_012802).

Single Cell RNA Sequencing (scRNAseq) of Transplanted mT3 Tumors and Data Analysis

mT3 tumor was generated by injecting 25,000 mT3 cells (derived from a PDAC of a KPC C57BL/6 mouse subcutaneously into the back flank of 10-week-old female C57BL/6 mice in a 1:1 suspension of Matrigel (Cat #354234, Corning) and PBS. At 3 weeks post injection, the tumor was dissected and processed as described before to obtain single cell suspensions. Subsequently, immune cells and blood cells were removed by CD45+ magnetic bead-based depletion (Cat #130-052-301, Miltenyi Biotech) and ACK lysis buffer (Cat #A1049201, Gibco), respectively, following manufacturer's guidelines. Remaining cells were prepared for single cell sequencing using Chromium Single Cell 3′ GEM, Library & Gel Bead Kit v3 (Cat #1000075, 10× Genomics) on a 10× Genomics Chromium Controller following manufacturers protocol and sequenced using Illumina NextSeq 500 sequencer. The Cell Ranger Single-Cell Software Suite (10× Genomics) was used to perform sample demultiplexing, barcode processing, and single-cell 3′gene counting. Sequencing data was aligned to the mouse reference genome (mm10) using “cellranger mkfastq” with default parameters. Unique molecular identifier (UMI) counts were generated using “cellranger count”. Further analysis was performed in R using the Seurat package as described before. Briefly, cells with fewer than 500 detected genes per cell and genes that were expressed by fewer than 5 cells were filtered out. Subsequently, cells with >7800 genes were filtered out to remove noise from droplets containing more than one cell. Dead cells were excluded by retaining cells with <5% mitochondrial reads. The data was subsequently normalized by employing a global-scaling normalization method “LogNormalize” followed by identification of 2,000 most variable genes in the dataset, data scaling and subsequently dimensionality reduction by principal component analysis (PCA) using the 2000 variable genes. Then, a graph based clustering was performed using Louvain algorithm implemented in Seurat and clusters of cells with distinct gene expression profiles were identified. A non-linear dimensional reduction was then performed via uniform manifold approximation and projection (UMAP) and the clusters expressing Pdpn, Pdgfra, Thy1 and Dcn markers were identified as CAFs. All animal experimental procedures were completed under an approved IACUC protocol at LLNL and conforming to the NIH Guide for the care and use of laboratory animals.

Analysis of Cancer-Associated Fibroblasts from Mouse Pancreatic Tumors

scRNAseq data from PDAC tumors of 2 KPC mice were obtained from GEO (GSE114417) (Biffi et al. Cancer Discov. CD-18-0710 (2018)) as log transformed gene-by-cell count matrix. Subsequent data analysis was carried out in R with Seurat (Stuart and Butler, Cell 177: 1888-1902 (2019)) as described above, and various cell types were identified. Clusters of cells expressing CAF markers Pdgfra, Pdpn, Thy1 and Col1a1 were identified as CAFs.

### Statistical Analysis

Statistical tests were performed using GraphPad Prism 5 software (GraphPad Software, La Jolla Calif. USA, www.graphpad.com; RRID: SCR_002798), as indicated in each figure. Each figure specifies number of individual data points. Mean and standard error of the mean are shown.

### Results

### CDH11 is Increased in the Stroma but not the Epithelial or Immune Component of Pancreatitis and Pancreatic Cancer Patients, Mouse Models and Cells

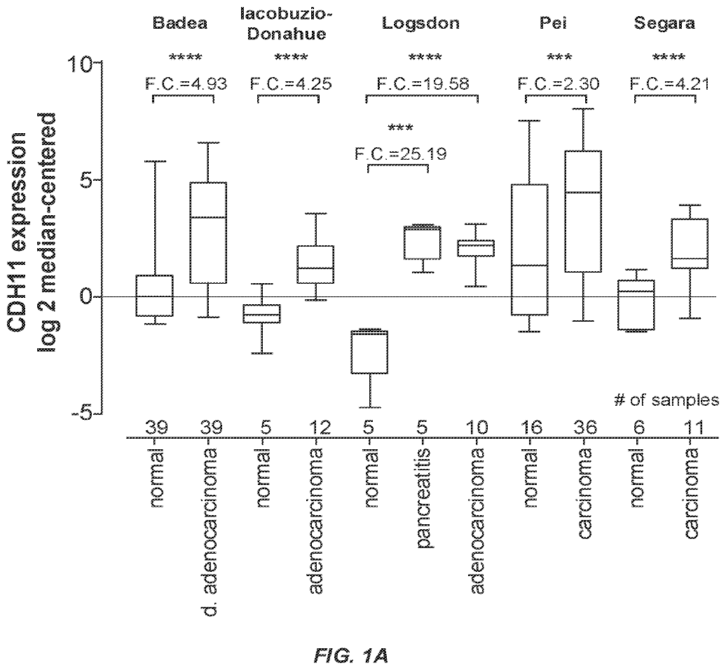

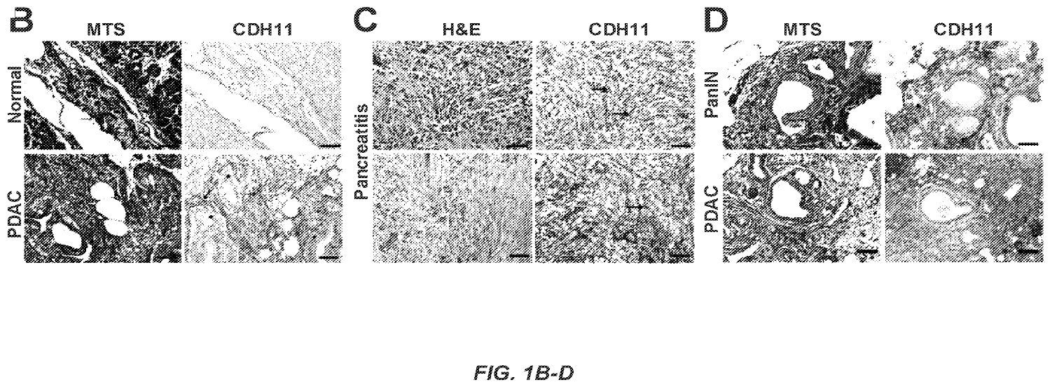

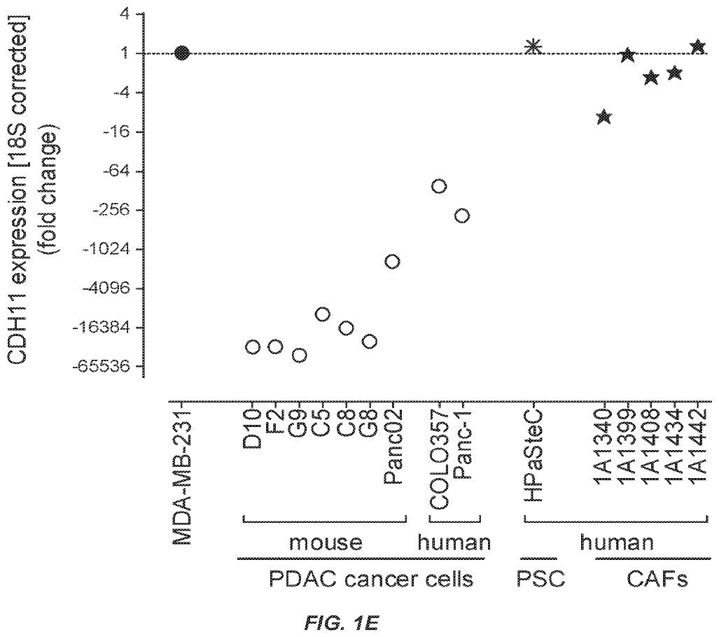

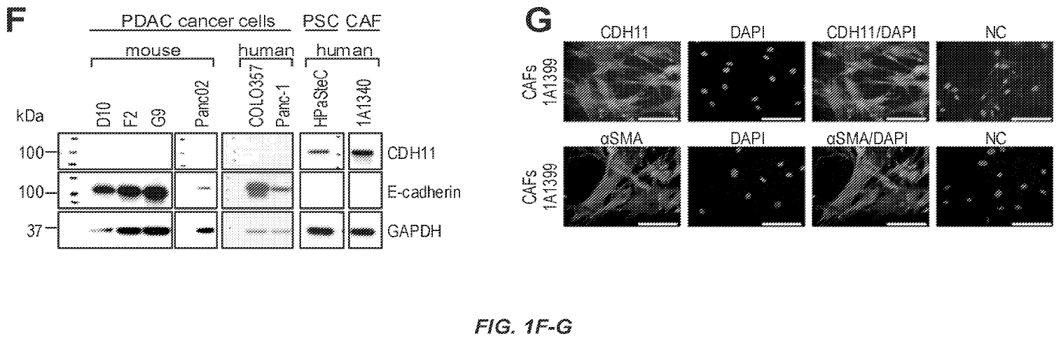

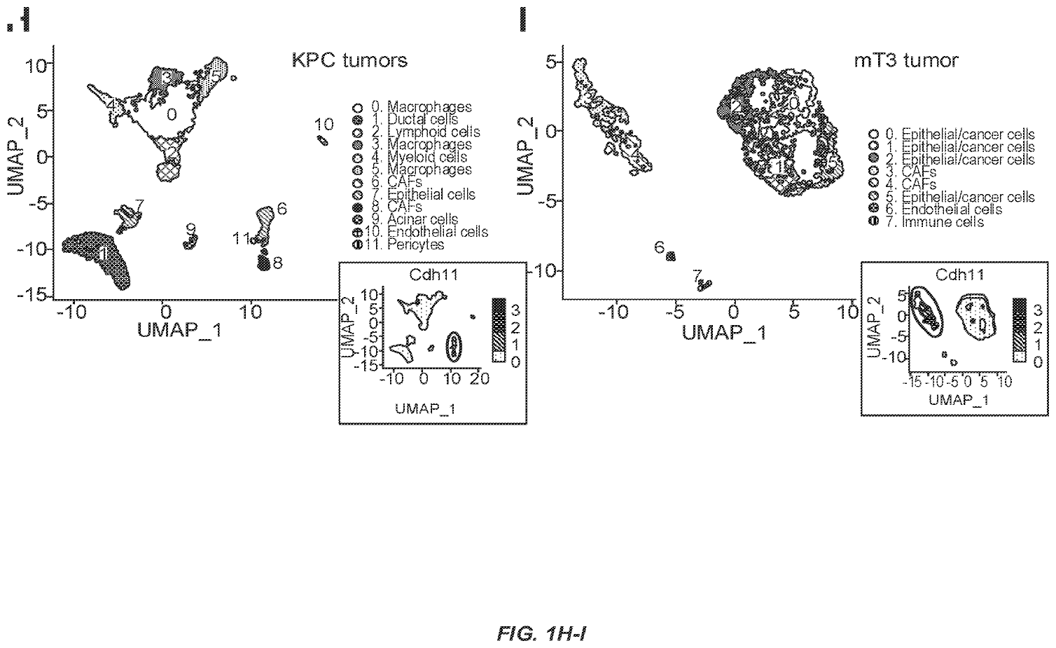

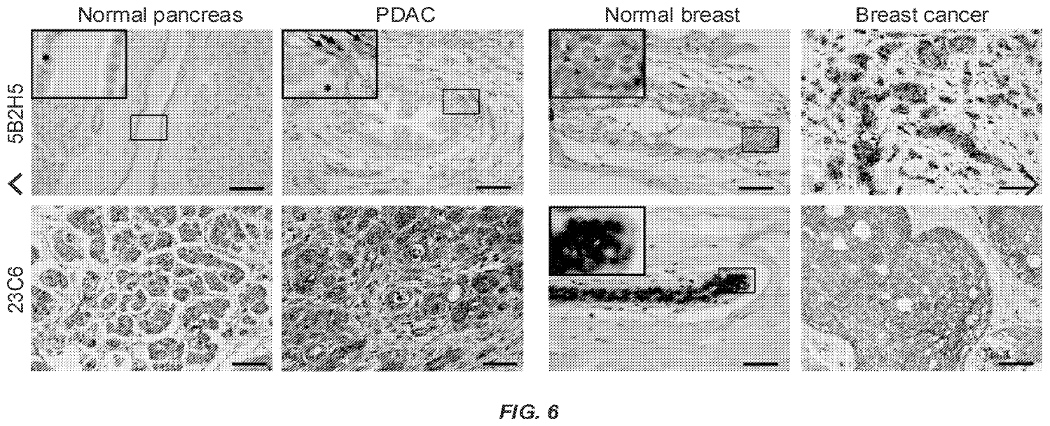

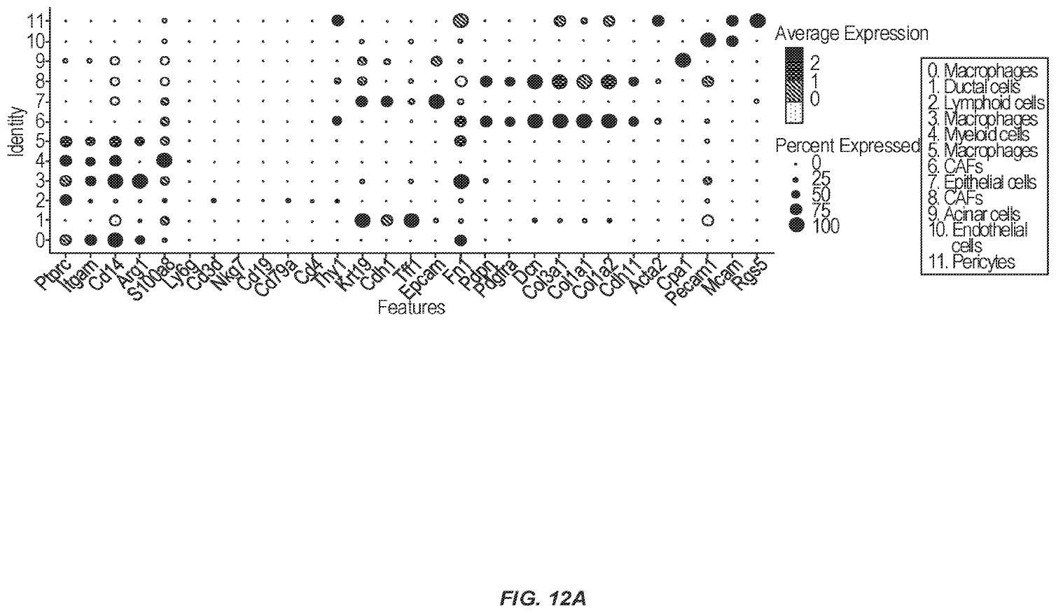

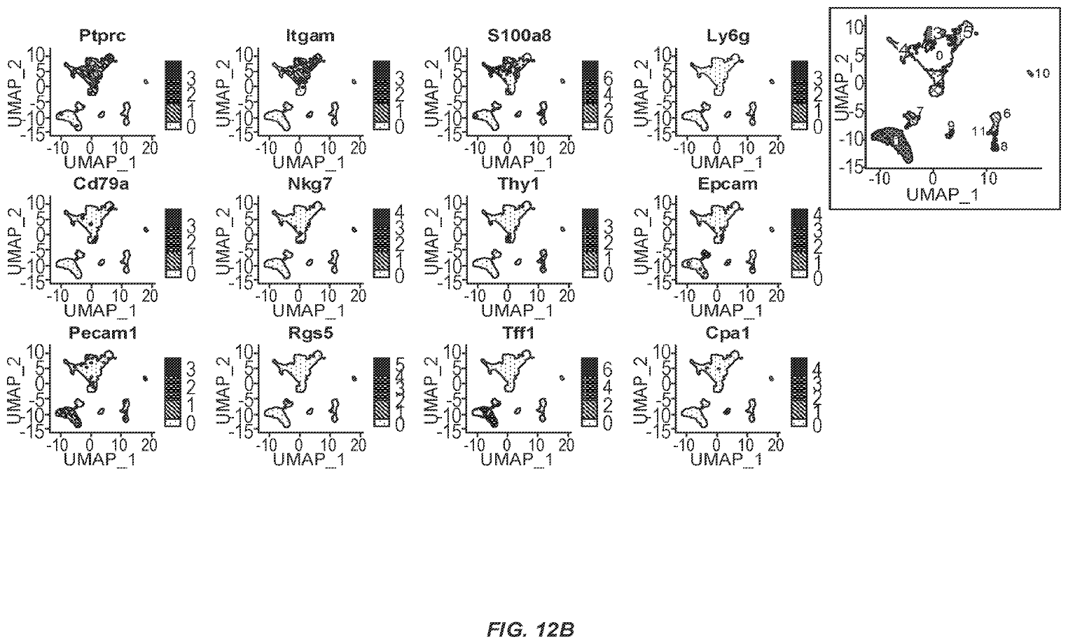

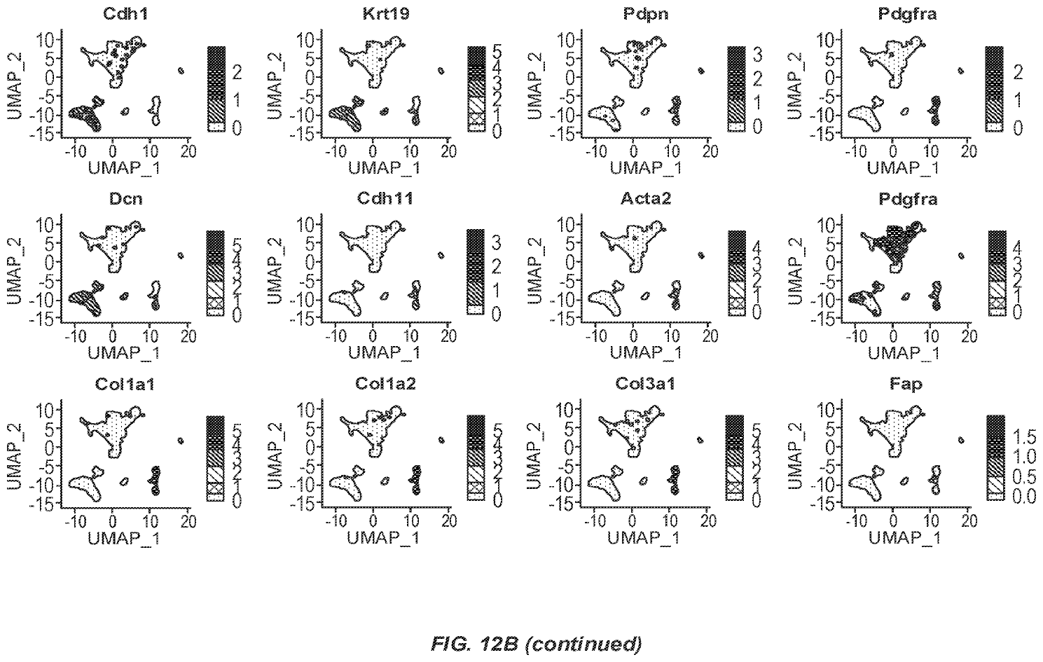

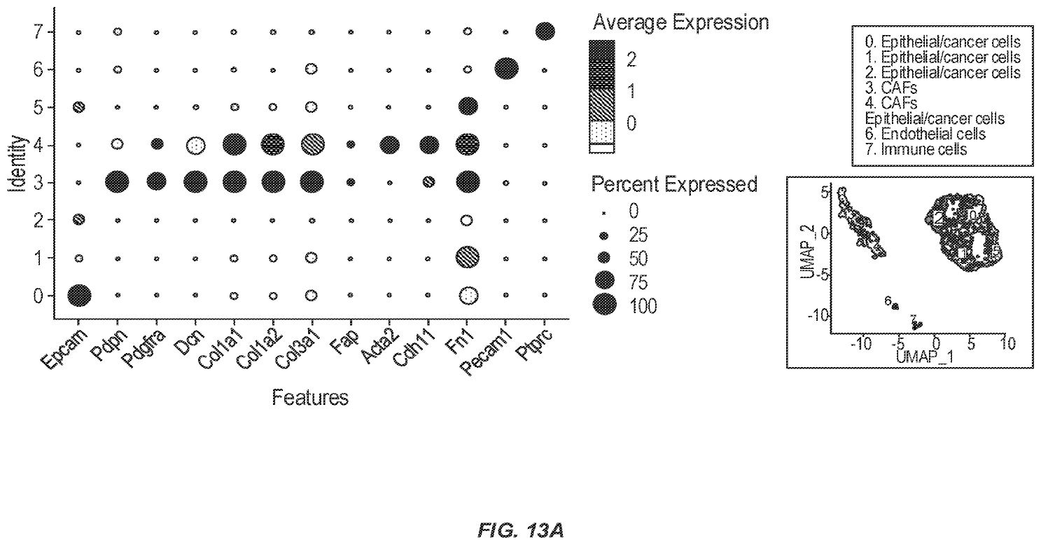

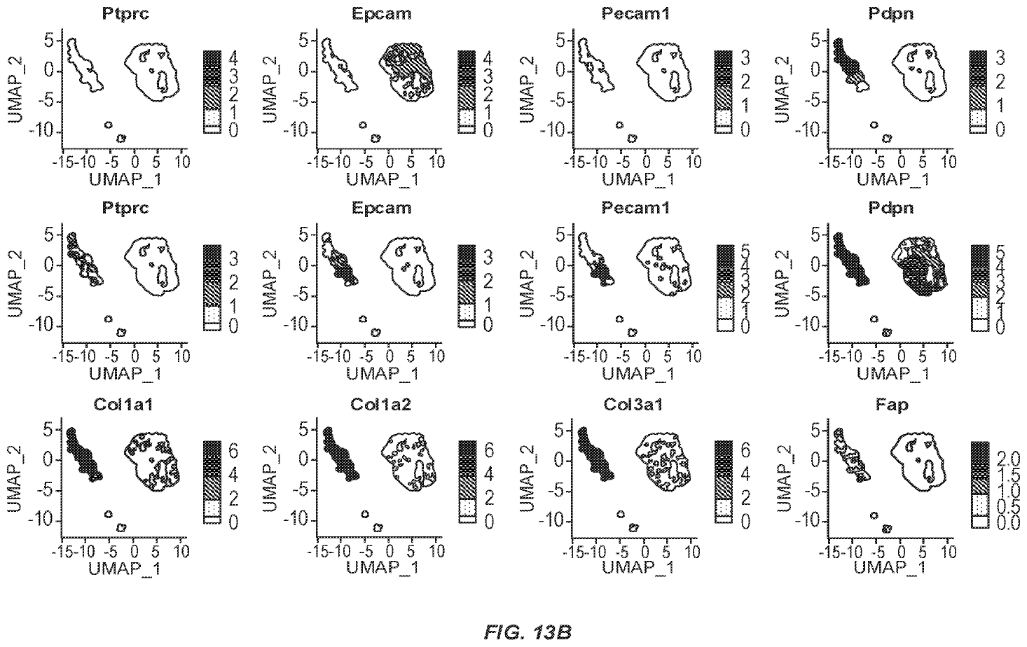

CDH11 transcripts are increased up to 25-fold in patients with pancreatitis and pancreatic cancer when compared to normal pancreas (FIG. 1A, Oncomine data extracted from Badea et al. Hepatogastroenterology 55:2016-2027 (2008); Iacobuzio-Donahue, et al. Am J Pathol 162:1151-1162 (2003); Logsdon et al. Cancer Res 63:2649-2657 (2003); Pei H et al. Cancer Cell 16:259-266 (2009); Segara et al. Clin Cancer Res 11:3587-3596 (2005)). Activated stroma surrounding human and mouse pancreatic lesions stained positive for CDH11 protein, while the epithelial pancreatic cancer cells remain negative when detected by 5B2H5 antibody—specific for native and denatured CDH11 (FIG. 1B-D). FIG. 6 explains discrepancies with another study showing CDH11 expression in cancer epithelial cells in addition to activated PSCs (Birtolo et al. Am J Pathol 187:146-155 (2017)). These data suggest that the 23C6 antibody used in that study, while raised against and specific for native CDH11, recognizes other cadherins in their denatured state (in IHC and WB). Next, CDH11 mRNA expression was assessed across different human and mouse pancreatic cancer cell lines and primary CAFs isolated from human PDAC, to support further use of the 5B2H5 antibody throughout the study. In human and mouse, CDH11 mRNA expression was 10-10,000-fold higher in CAFs than in pancreatic cancer epithelial cells (FIG. 1E). Similarly, human primary CAFs express high levels of CDH11 protein, while a panel of human and mouse pancreatic cancer epithelial cell lines express very low (usually undetectable) levels of CDH11, but high levels of E-cadherin (FIG. 1F). The activation status of human CAFs was confirmed by positive staining for alpha smooth muscle actin (αSMA) (FIG. 1G). Furthermore, single cell RNA sequencing (scRNAseq) data derived from a Kras^{+/LSL-G12D};Trp53^{+/LSL-R172H};Pdx-Cre PDAC mouse model (GSE114417), showed that CDH11 is primarily expressed by CAFs, with negligible expression in other cell types including cancer epithelial cells or immune cells (FIG. 1H and FIGS. 12A-B). Additionally, tumors were generated in immunocompetent C57BL/6J mice using the PDAC cell line, mT3, derived from the Kras^{+/LSL-G12D};Trp53^{+/LSL-R172H};Pdx-Cre C57BL/6J mouse model (Boj et al. Cell 160:324-338 (2015). Upon scRNAseq analysis of immune-cell-depleted tumors, we detected CDH11 expression almost exclusively in two CAF subpopulations, with virtually no expression in cancer epithelial cells (FIG. 1I, FIGS. 13A-B). These data indicate that in the context of pancreatic cancer, CAFs are the predominant cell type that produces CDH11.

### Genetic Targeting of CDH11 Significantly Improves Survival in KPC Mouse Model

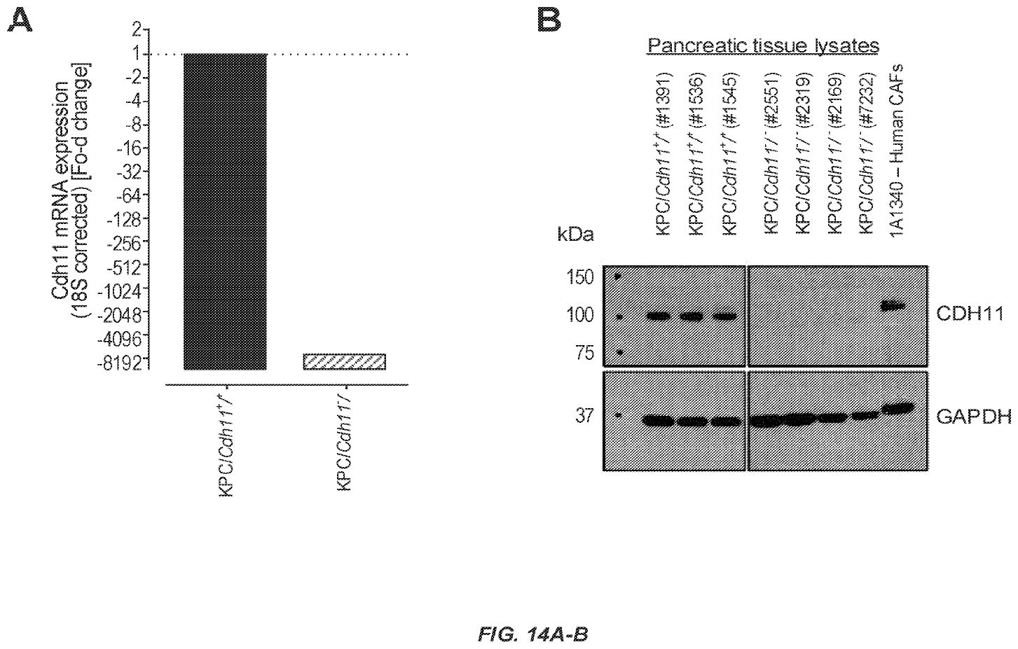

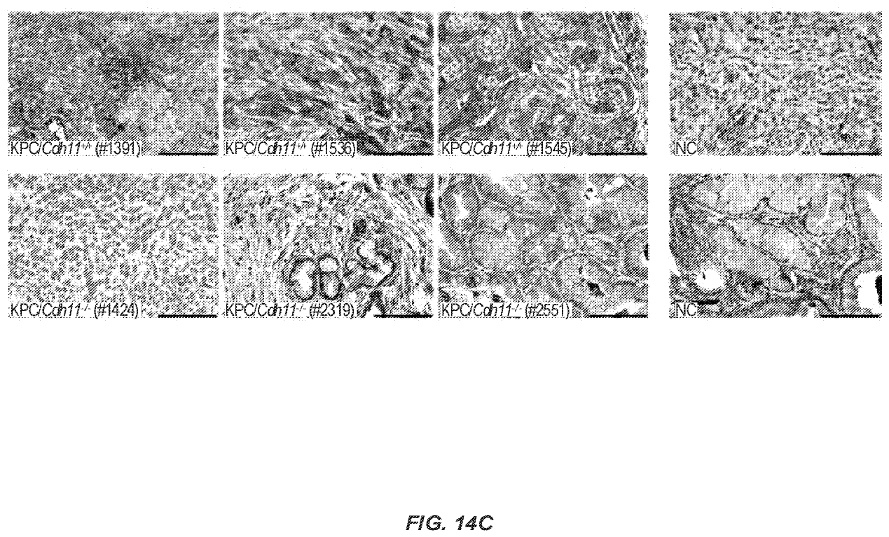

To study the role of CDH11 in PDAC, an immunocompetent p48-Cre;LSL-Kras^{G12D/+};LSL-Trp53^{R172H/+} (KPC) mouse model that resembles progression of human disease was used. By introducing the knockout CDH11 allele (CDH11^{-/-}), three KPC genotypes, with different CDH11 status (KPC/CDH11^{+/+}, KPC/CDH11^{+/-} and KPC/CDH11^{-/-}), were produced (FIGS. 14A-C).

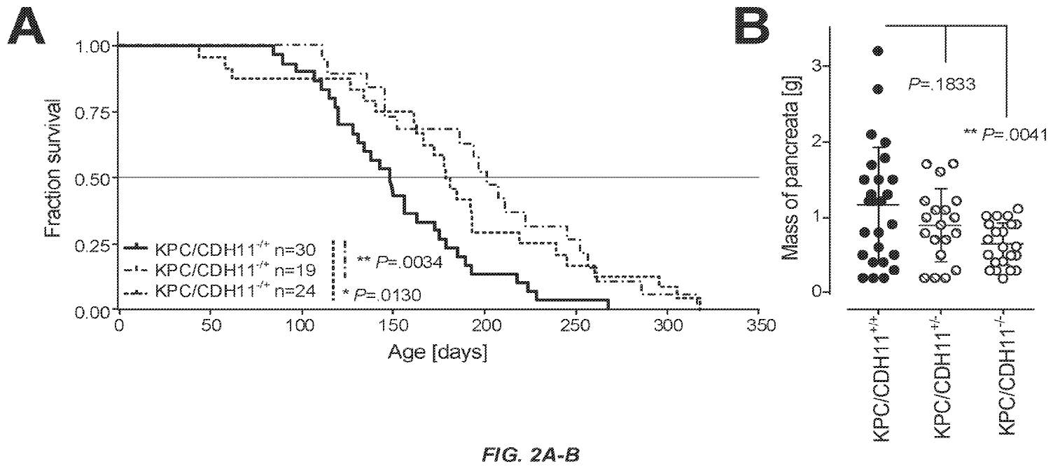

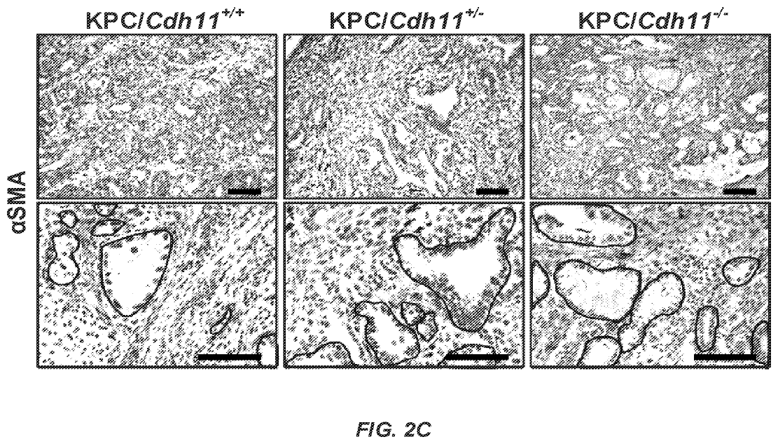

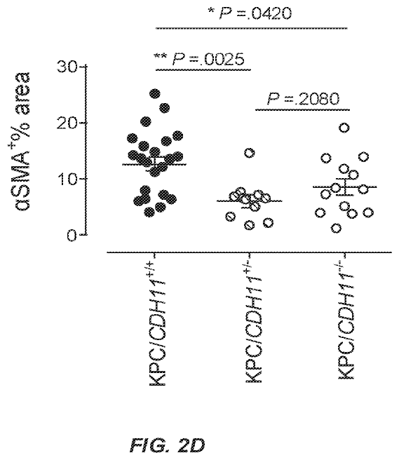

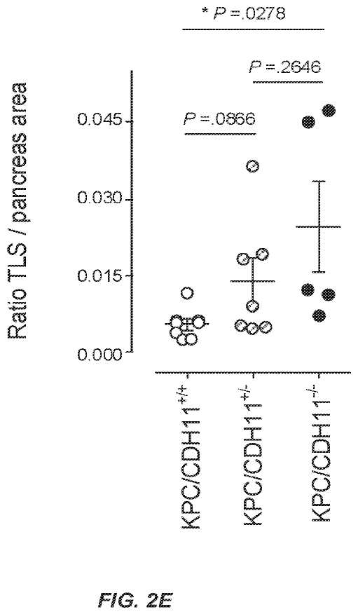

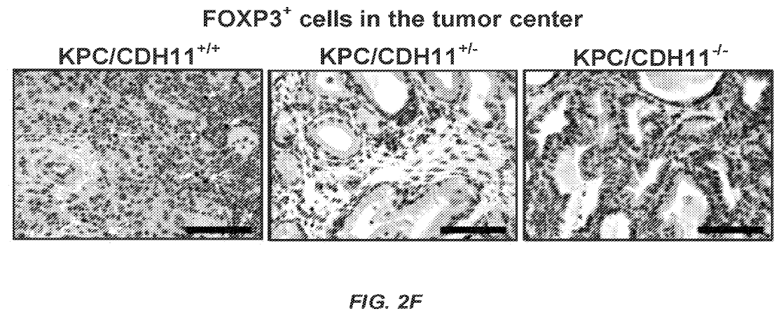

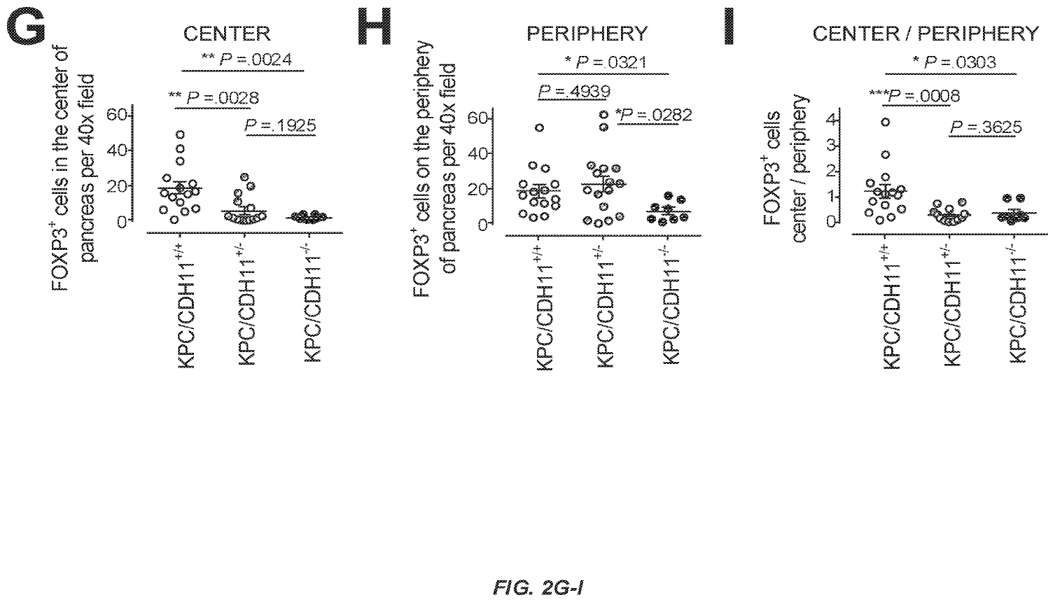

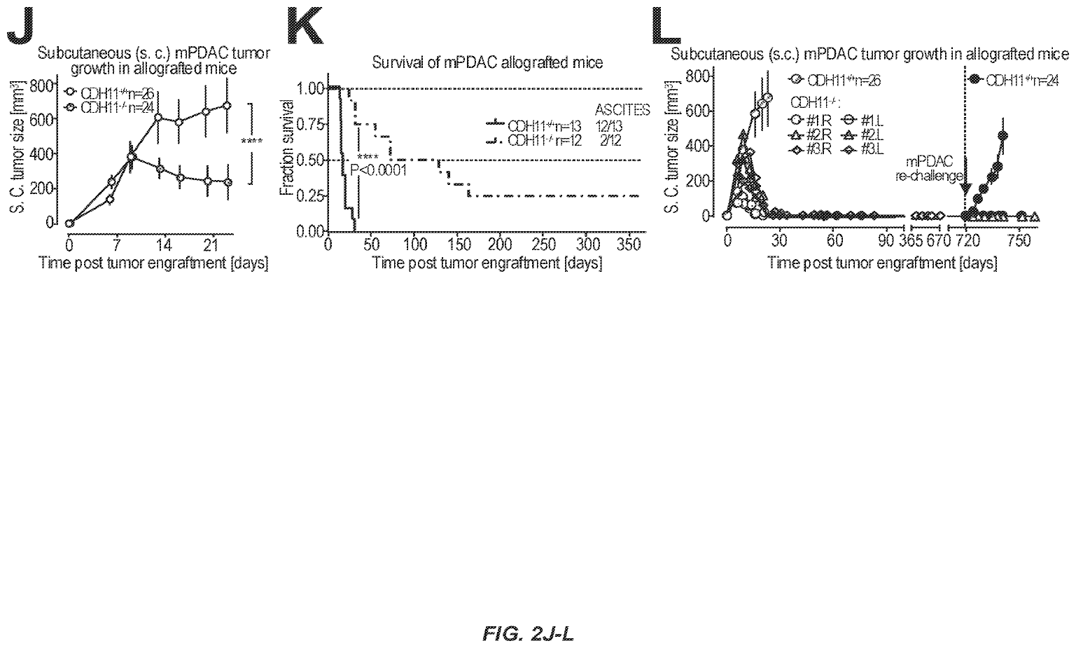

Strikingly, mice with zero or one allele of CDH11 survived significantly longer than mice with both wild-type copies of CDH11 (FIG. 2A). Moreover, at the time of euthanasia, tumors from the KPC/CDH11^{+/+} animals were significantly larger (1.2 g) than those from KPC/CDH11^{-/-} mice (0.6 g) (FIG. 2B), even though the rate of macrometastases in KPC/CDH11^{+/+} mice (82%) was similar to KPC/CDH11^{+/-} (79%) and KPC/CDH11^{-/-} mice (79%), and to the macrometastasis rate of 76% shown by Hingorani et al. (Cancer Cell 7:469-483 (2005)).

### Targeting CDH11 Modulates the Tumor Microenvironment

### CDH11 Deficiency Affects the Pattern of Fibrosis in the KPC Mouse Model

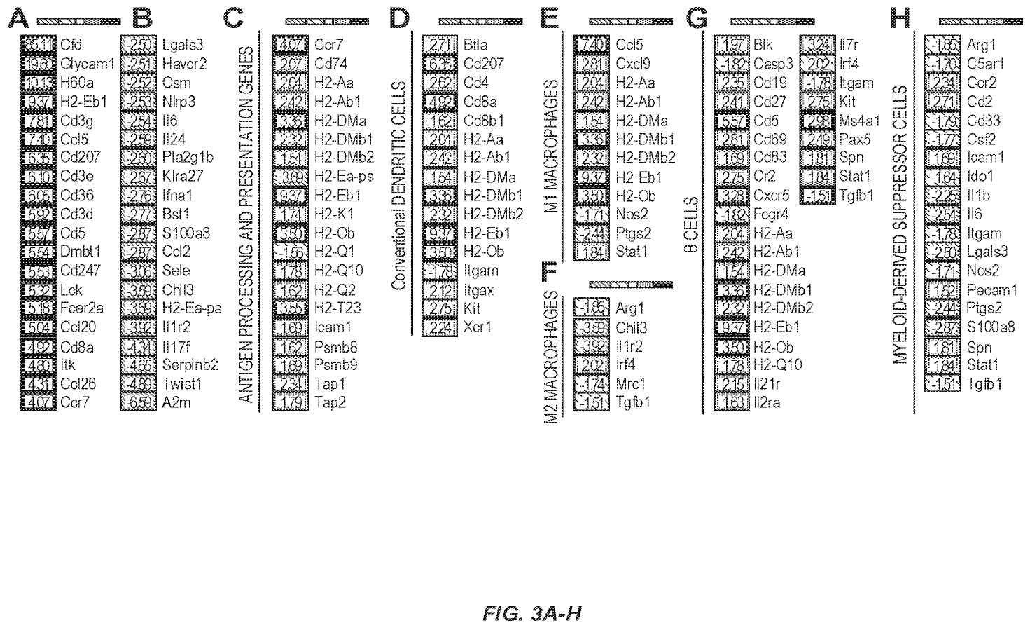

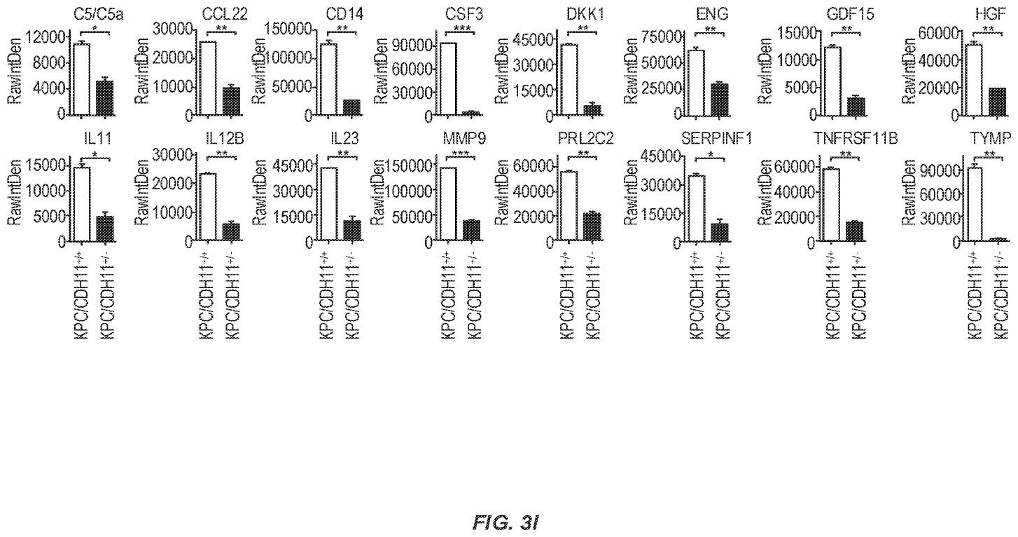

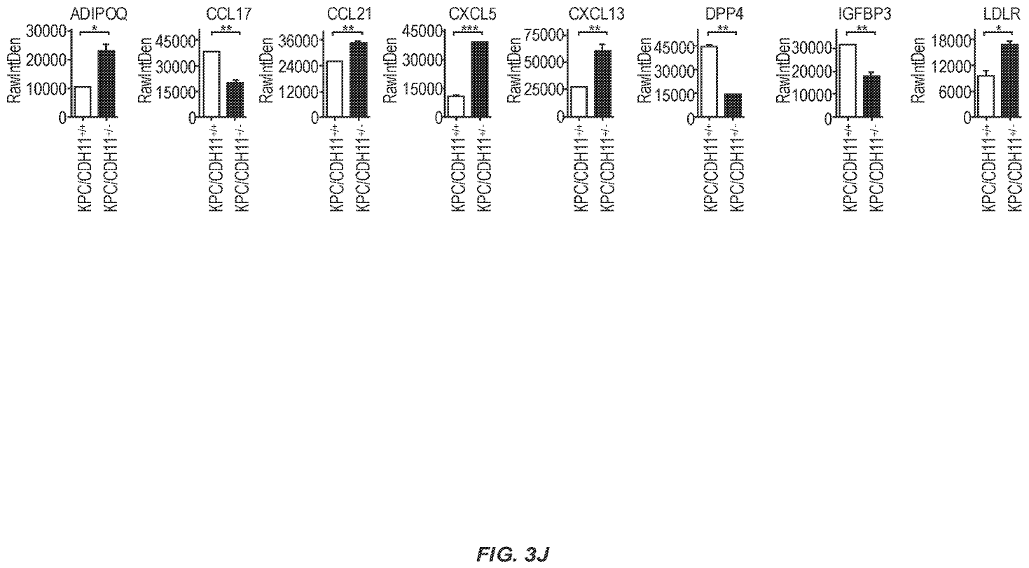

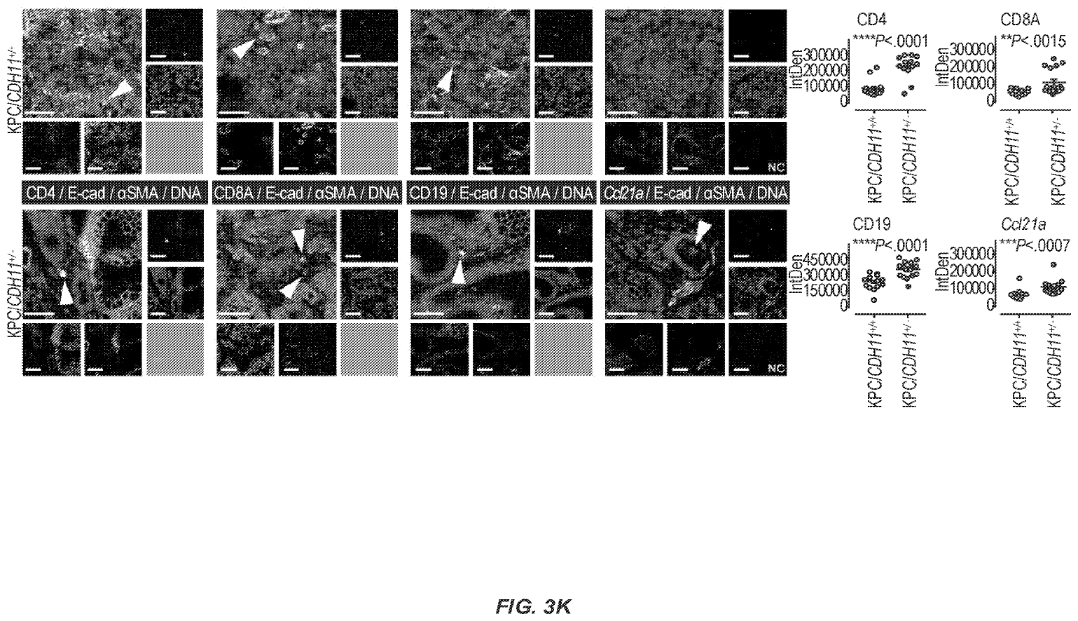

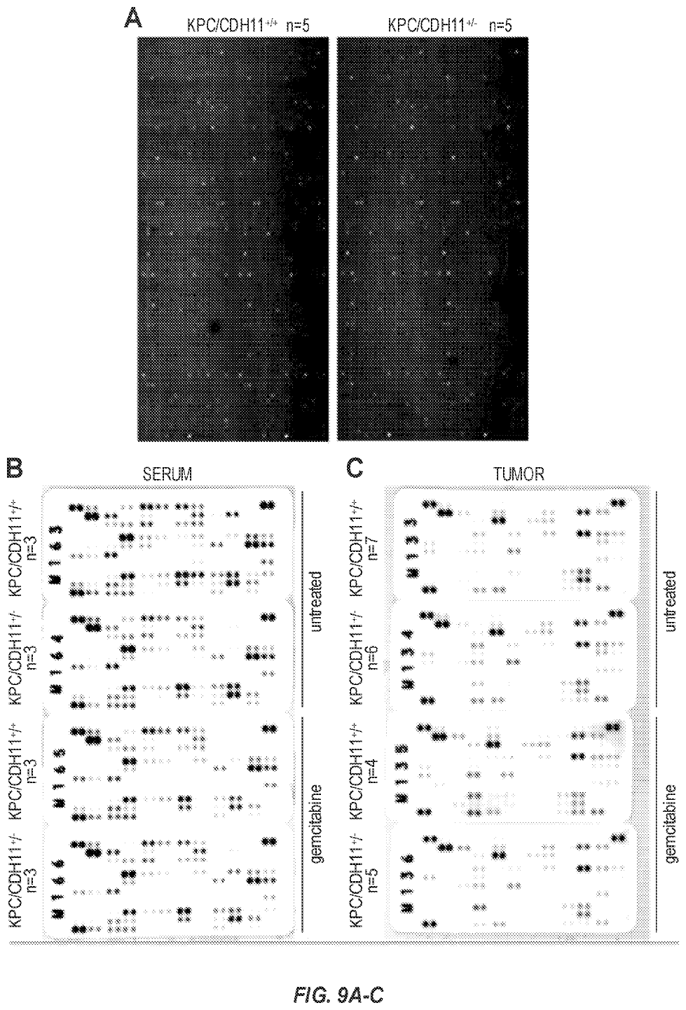

To assess correlation between stromal activation and CDH11 status, the pattern of αSMA expression was analyzed by IHC and RNAseq. Collagen content was analyzed by gene expression analyses. In the KPC model, a difference in stromal activation assessed by αSMA⁺ IHC staining was clearly apparent around early pancreatic intraepithelial neoplasms (PanIN). Already, at the PanIN1/2 stage, KPC/CDH11^{+/+} mice exhibited substantial stromal activation around every lesion, while KPC/CDH11-deficient animals displayed incomplete encirclement of the lesions (FIG. 2C). Quantification of αSMA⁺ staining (FIG. 2D) was confirmed by RNAseq analysis of pancreata collected at the time of euthanasia, showing ˜2.5-fold decrease in Acta2 (αSMA) expression in KPC/CDH11^{+/-} mice compared to KPC/CDH11^{+/+}. Additionally, the anticipated 2.45-fold decrease in CDH11 transcripts of KPC/CDH11^{+/-} pancreata in comparison to KPC/CDH11^{+/+} correlated with ˜2-3-fold reduced expression of multiple collagens and fibronectin. Finally, more tertiary lymphoid structures were detected (TLSs) in pancreata of KPC/CDH11-deficient mice (FIG. 2E), together with an increase in TLS-promoting cytokines Ccl21, Cxcl13 and Ccl19, and their receptors Ccr7 and Cxcr5, when compared to KPC/CDH11^{+/+} (FIGS. 3A and J, FIG. 9, Table 2).

### CDH11-Deficiency Reduces the Number of T-Regs Infiltrating Center of PDAC Tissue

Next, the distribution of FOXP3⁺ T-reg cells within the PDAC tissue of KPC mice (FIG. 2F-I) was analyzed. The total number of FOXP3⁺ cells (tumor center+ periphery) was similar in the KPC/CDH11^{+/+} and KPC/CDH11^{+/-} groups, but was significantly reduced in KPC/CDH11^{-/-} mice. However, a marked reduction in the number of immunosuppressive FOXP3⁺ cells in the center of tumors (>300 μm from the tissue margin) from KPC/CDH11-deficient animals (FIGS. 2G and I) correlated with improved survival (FIG. 2A). Additionally, the FOXP3⁺ cell infiltration of the tumor center positively correlated with tissue and serum expression of CCL17 and CCL22, known to attract T-regs (FIG. 3I-J, Table 2, FIG. 9B-C).

### CDH11^{-/-} Mice Reject Allograft Tumors and Induce Immune Memory

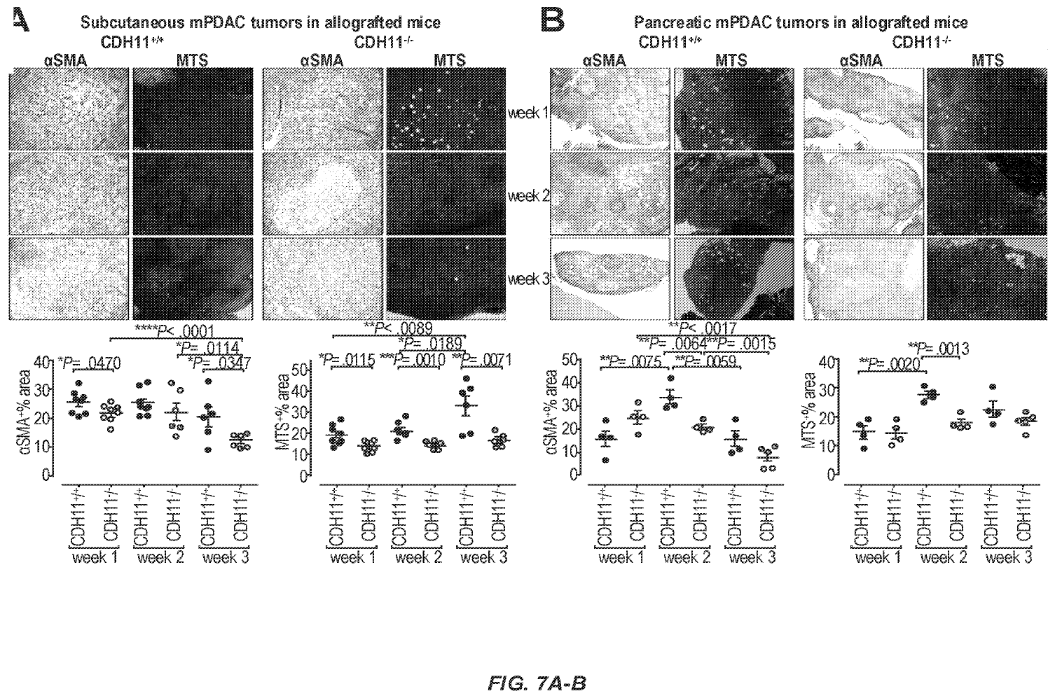

In an additional series of experiments, mouse pancreatic cancer cells (mPDAC, clone G8), isolated from the pancreatic cancer of a KPC mouse, were inoculated into CDH11^{+/+} and CDH11^{-/-} animals. The CDH11 colony was bred into the background of the KPC colony for multiple generations to produce CDH11^{+/+} and CDH11^{-/-} littermates on the same mixed background as the mPDAC G8 cells. Thirteen CDH11^{+/+} and twelve CDH11^{-/-} mice were s.c. and i.p. engrafted with mPDAC cells. Initial s.c. tumor growth was comparable in CDH11^{+/+} and CDH11^{-/-} animals. However, approximately 10-14 days after mPDAC cell injection, s.c. tumors (with average volume of ˜400 mm³) in many CDH11^{-/-} animals started regressing (FIG. 2J). The median survival of engrafted CDH11^{+/+} mice was only 17 days, and all 13 mice succumbed to the disease by one month after mPDAC engraftment. CDH11^{-/-} mice survived significantly longer (101 days on average), and ascites developed in only 17% of animals, c.f. 92% of CDH11^{+/+} mice (FIG. 2K). Furthermore, quantification of αSMA⁺ IHC and MTS staining showed decreased stromal activation and no excess collagen deposition in CDH11 mice compared to CDH11^{+/+} littermates during the 3-week time period after mPDAC cell engraftment (FIG. 7). Finally, three CDH11^{-/-} mice that initially grew large s.c. tumors (˜100-500 mm³), lost tumors entirely without sign of recurrence for >1.7 year. Two of them survived longer than 2 years, and were re-challenged with the same mPDAC cell line by s.c. injection. Upon re-challenge, these CDH11^{-/-} mice did not develop any tumors (FIG. 2L), suggesting the presence of immune memory long after the initial challenge. At the same time, CDH11^{+/+} animals formed large tumors with the same mPDAC cell preparation. Although we acknowledge that this model is not truly syngeneic even after backcrossing, only CDH11^{-/-} mice lost their tumors, while none of their CDH11^{+/+} littermates did.

### CDH11-Deficiency Induces an Anti-Tumor Immunity and Reduces Immunosuppression

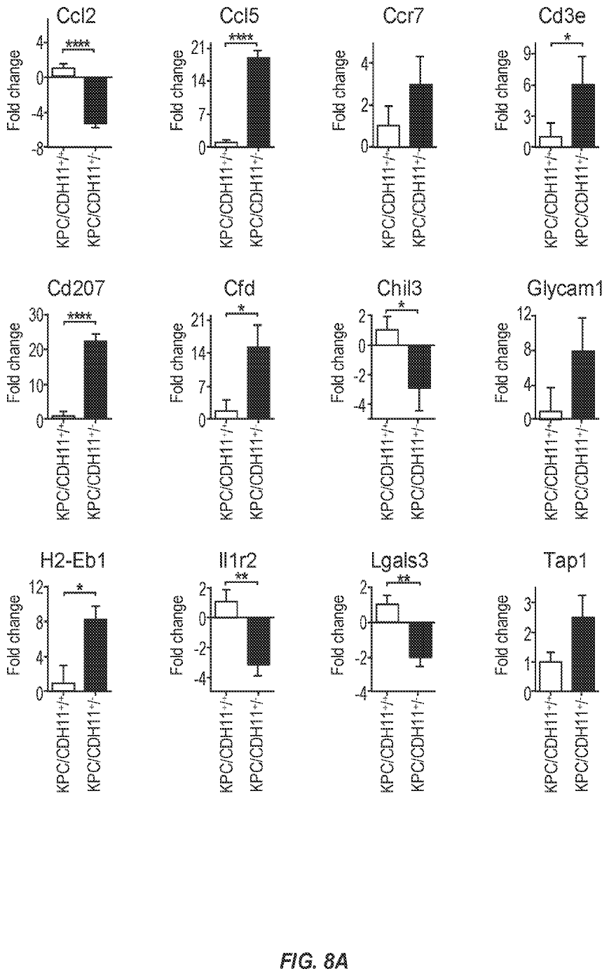

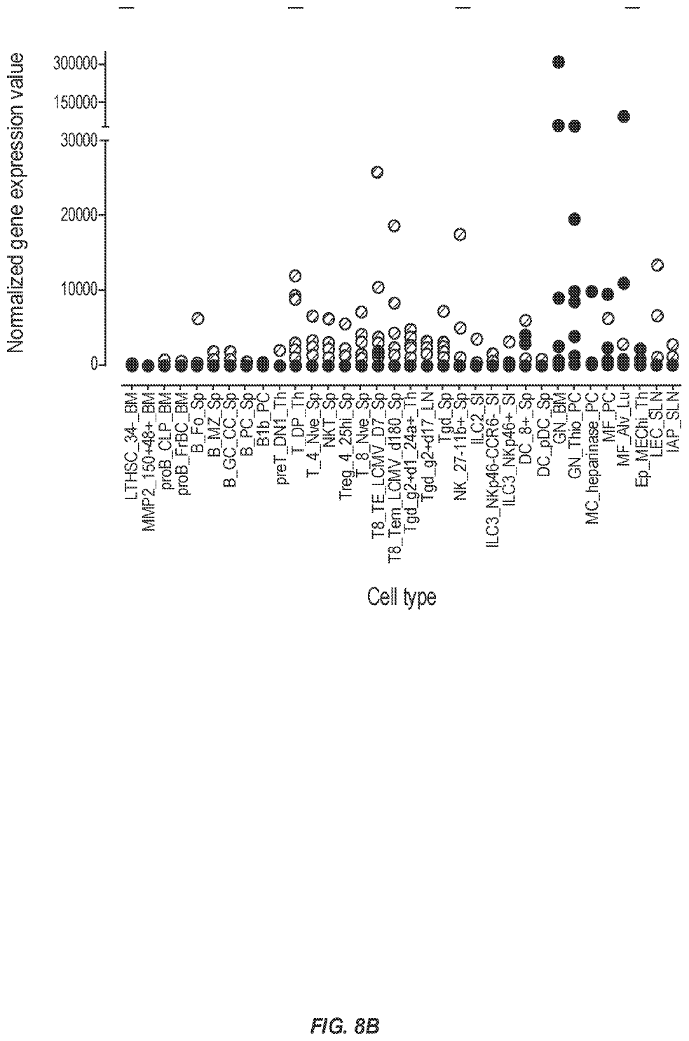

To achieve deeper insight into the KPC TME, gene expression of pooled pancreatic tumors isolated from KPC/CDH11^{+/-} mice at the time of euthanasia, normalized to KPC/CDH11^{+/+} by Nanostring (FIG. 3A-H), was analyzed. The Nanostring data was confirmed by qPCR reactions on each pancreatic sample (FIG. 8), and RNAseq, in addition to cytokine protein expression (FIG. 1-K, FIG. 9B-C, Table 2).

Compared to KPC/CDH11^{+/+}, in pancreata of KPC/CDH11^{+/-} mice increased expression of multiple antigen processing and presentation genes, T cell receptor (TCR) components, and kinases downstream of TCR (Lck, Itk and Zap70) was detected (FIGS. 3A and C). Furthermore, phosphorylation of PKC-θ and SLP-76, involved in TCR downstream signaling (Table 1, FIG. 9A) was increased in KPC/CDH11^{+/-} tumors. Additionally, markers associated with conventional dendritic cells (cDC), M1 macrophages, and B cells were increased in KPC/CDH11^{+/-} tumors (FIGS. 3D-E and G), as well as cytokines (mRNA and protein) that attract B, T, and dendritic cells: CXCL13, CCL21 and CCL19 (FIG. 3J, Table 2, FIG. 9C). Furthermore, in KPC/CDH11^{+/-} PDAC tissues, phosphorylation of mTOR and PI3K was decreased (Table 1, FIG. 9A), along with reduced expression of M2 macrophage, MDSC and other immunosuppressive markers (FIGS. 3B, F and H). The Skyline database for the top 20 increased or decreased genes from the KPC/CDH11^{+/-} tumors (FIG. 3A-B, FIG. 8B) was queried, and it was found that many of the up-regulated genes are associated predominantly with T cells, and some with cDCs, B cells, and NK cells. In contrast, the decreased genes are mainly associated with cells that share immunosuppressive properties: macrophages, monocytes and neutrophils. Many soluble factors that support infiltration by immunosuppressive cells, such as CSF3, DKK1, CD14, IL11, C5/C5a, CCL17, CCL22, etc. (FIG. 3I-J, Table 2, FIG. 9B-C) were decreased. Additionally, changes in ADIPOQ and LDLR protein levels (FIG. 3J, FIG. 9, Table 2) indicate altered lipid metabolism. The Ingenuity Pathway Analysis of RNAseq data also indicates CDH11-dependent changes in lipid metabolism, extracellular matrix modification, fibrosis and stellate cell activation, agranulocyte and granulocyte diapedesis, and others (Table 3).

TABLE 1

| | | Fold change |
|---|---|---|
| | | (KPC/CDH11^{+/-}/ |
| Antibody list | | KPC/CDH11^{+/+}) |
| Phospho protein | Total protein | [untreated] |
| PKC theta (Phospho-Ser676) | PKC theta (Ab-676) | 1.48 |
| Estrogen Receptor-alpha (Phospho-Ser106) | Estrogen Receptor-alpha (Ab-106) | 1.45 |
| SLP-76 (Phospho-Tyr128) | SLP-76 (Ab-128) | 1.45 |
| Lamin A/C (Phospho-Ser392) | Lamin A/C (Ab-392) | 1.42 |
| Estrogen Receptor-alpha (Phospho-Ser104) | Estrogen Receptor-alpha (Ab-104) | 1.39 |
| AMPK1/AMPK2 (Phospho-Ser485/491) | AMPK1/AMPK2 (Ab-485/491) | 1.39 |
| PLK1 (Phospho-Thr210) | PLK1 (Ab-210) | 1.36 |
| FAK (Phospho-Tyr397) | FAK (Ab-397) | 1.36 |
| Caspase 8 (Phospho-Ser347) | Caspase 8 (Ab-347) | 1.33 |
| p53 (Phospho-Ser6) | p53 (Ab-6) | 1.33 |
| Raf1 (Phospho-Ser296) | Raf1 (Ab-296) | 1.33 |
| GSK3 alpha (Phospho-Ser21) | GSK3 alpha (Ab-21) | 1.32 |
| JunD (Phospho-Ser255) | JunD (Ab-255) | 1.31 |
| IKK-alpha/beta (Phospho-Ser180/181) | IKK-alpha/beta (Ab-180/181) | −1.31 |
| HER2 (Phospho-Tyr877) | HER2 (Ab-877) | −1.31 |
| Smad3 (Phospho-Ser204) | Smad3 (Ab-204) | −1.32 |
| Pyk2 (Phospho-Tyr881) | Pyk2 (Ab-881) | −1.32 |
| DARPP-32 (Phospho-Thr34) | DARPP-32 (Ab-34) | −1.33 |
| Smad3 (Phospho-Ser213) | Smad3 (Ab-213) | −1.33 |
| Opioid Receptor (Phospho-Ser375) | Opioid Receptor (Ab-375) | −1.33 |
| IKK-gamma (Phospho-Ser85) | IKK-gamma (Ab-85) | −1.33 |
| Shc (Phospho-Tyr349) | Shc (Ab-349) | −1.33 |
| VEGFR2 (Phospho-Tyr1054) | VEGFR2 (Ab-1054) | −1.33 |
| VASP (Phospho-Ser157) | VASP (Ab-157) | −1.33 |
| P70S6K-beta (Phospho-Ser423) | P70S6K-beta (Ab-423) | −1.34 |
| MEK1 (Phospho-Ser217) | MEK1 (Ab-217) | −1.35 |
| PLC beta3 (Phospho-Ser1105) | PLC beta3 (Ab-1105) | −1.35 |
| p27Kip1 (Phospho-Ser10) | p27Kip1 (Ab-10) | −1.35 |
| CREB (Phospho-Ser121) | CREB (Ab-121) | −1.37 |
| IkB-beta (Phospho-Thr19) | IkB-beta (Ab-19) | −1.39 |
| VAV2 (Phospho-Tyr142) | VAV2 (Ab-142) | −1.41 |
| Rb (Phospho-Ser795) | Rb (Ab-795) | −1.45 |
| Rho/Rac guanine nucleotide | Rho/Rac guanine nucleotide | −1.45 |
| exchange factor 2 (Phospho-Ser885) | exchange factor 2 (Ab-885) | |
| Vinculin (Phospho-Tyr821) | Vinculin (Ab-821) | −1.45 |
| FLT3 (Phospho-Tyr599) | FLT3 (Ab-599) | −1.46 |
| NMDAR1 (Phospho-Ser897) | NMDAR1 (Ab-897) | −1.46 |
| IR (Phospho-Tyr1361) | IR (Ab-1361) | −1.47 |
| JNK1/2/3 (Phospho-Thr183/Tyr185) | JNK1/2/3 (Ab-183/185) | −1.49 |
| HRS (Phospho-Tyr334) | HRS (Ab-334) | −1.52 |
| LAT (Phospho-Tyr191) | LAT (Ab-191) | −1.56 |
| PI3-kinase p85-subunit alpha/gamma | PI3-kinase p85-subunit | −1.62 |
| (Phospho-Tyr467/Tyr199) | alpha/gamma (Ab-467/199) | |
| mTOR (Phospho-Thr2446) | mTOR (Ab-2446) | −1.80 |

TABLE 2

| SERUM | | TUMOR | |
|---|---|---|---|
| | Fold change | | Fold change |
| | (KPC/CDH11^{+/-}/ | | (KPC/CDH11^{+/-}/ |
| | KPC/CDH11^{+/+}) | | KPC/CDH11^{+/+}) |
| Cytokine | [untreated] | Cytokine | [untreated] |
| CST3 | −1.31 | CXCL5 | 3.60 |
| VCAM1 | −1.31 | ADIPOQ | 2.12 |
| CD93 | −1.34 | IL1A | 1.85 |
| RBP4 | −1.37 | CXCL13 | 1.77 |
| NPTX2 | −1.51 | LDLR | 1.73 |
| IGFBP5 | −1.66 | IGFBP2 | 1.68 |
| AGER | −1.86 | FGF1 | 1.61 |
| IL33 | −1.88 | VEGFA | 1.45 |
| ENG | −2.07 | SERPINE1 | 1.39 |
| C5/C5a | −2.10 | CCL21 | 1.32 |
| HGF | −2.63 | IL33 | −1.31 |
| PRL2C2 | −2.64 | REG3G | −1.34 |
| CCL22 | −2.69 | CCL11 | −1.35 |
| IL11 | −3.01 | CST3 | −1.44 |
| SERPINF1 | −3.58 | IGFBP3 | −1.73 |
| MMP9 | −3.61 | CCL17 | −1.87 |
| IL23 | −3.77 | DPP4 | −3.20 |
| TNFRSF11B | −3.91 | | |
| GDF15 | −4.03 | | |
| IL12B | −4.19 | | |
| CD14 | −4.70 | | |
| DKK1 | −8.20 | | |
| CSF3 | −27.34 | | |
| TYMP | −41.41 | | |
| SERUM | | TUMOR | |
| | Fold change | | Fold change |
| | (KPC/CDH11^{+/-}/ | | (KPC/CDH11^{+/-}/ |
| | KPC/CDH11^{+/+}) | | KPC/CDH11^{+/+}) |
| Cytokine | [gemcitabine] | Cytokine | [gemcitabine] |
| TYMP | 4.48 | CCL21 | 5.55 |
| ENG | 3.15 | IL33 | 3.64 |
| CD14 | 2.75 | CXCL13 | 2.55 |
| TNFRSF11B | 2.62 | IGFBP6 | 2.12 |
| IL4 | 2.62 | REG3G | 1.75 |
| CCL17 | 1.98 | FGF1 | 1.65 |
| F3 | 1.78 | GAS6 | 1.59 |
| RBP4 | 1.62 | DPP4 | 1.41 |
| MPO | 1.33 | CHIL1 | −1.69 |
| IL33 | 1.31 | CCN4 | −1.86 |
| LCN2 | −1.44 | CFD | −1.88 |
| PRL2C2 | −1.49 | IL1A | −2.04 |
| IL11 | −1.61 | CXCL16 | −2.08 |
| CCL12 | −1.68 | CRP | −2.11 |
| NPTX2 | −1.68 | IGFBP2 | −2.24 |
| ANGPT1 | −1.72 | SERPINE1 | −4.09 |
| AGER | −1.85 | IGFBP3 | −5.72 |
| PDGFB | −3.56 | LDLR | −5.95 |
| | | PTX3 | −6.18 |
| | | VEGFA | −7.17 |
| | | POSTN | −7.22 |
| | | IL11 | −10.46 |

TABLE 3
RNAseq - differentially expressed genes in KPC/CDH11^{+/-} versus KPC/CDH11^{+/+} [untreated]

| | −log(p- | |
|---|---|---|
| Ingenuity Canonical Pathways | value) | Molecules |
| FXR/RXR Activation | 6.92 | C4A/C4B, KNG1, APOE, PKLR, FETUB, AHSG, INS, NR5A2, APOC2, IL1F10, GC, Ins1 |
| Neuroprotective Role of THOP1 in | 6.23 | KNG1, HGFAC, DPP4, PRSS16, PRTN3, PRSS2, SST, CTRB2, FAP, HLA-E, PRSS3 |
| Alzheimer's Disease | | |
| Atherosclerosis Signaling | 5.09 | COL1A1, APOE, MMP3, CXCL12, MMP13, S100A8, APOC2, PNPLA3, IL1F10, |
| | | PDGFD |
| Inhibition of Matrix Metalloproteases | 4.94 | MMP3, MMP8, MMP10, MMP13, A2M, TFPI2 |
| Hepatic Fibrosis/Hepatic Stellate | 4.39 | IGFBP4, COL1A1, IGF2, COL6A3, MYH14, LAMA1, PDGFRA, EDNRA, |
| Cell Activation | | MMP13, PDGFD, A2M |
| Agranulocyte Adhesion and Diapedesis | 4.27 | MMP3, MYH14, MMP8, CXCL12, MMP10, MMP13, CXCL17, CLDN9, ACTG2, |
| | | IL1F10, CLDN6 |
| Maturity Onset Diabetes of Young | 3.82 | PKLR, INS, Ins1, PDX1 |
| (MODY) Signaling | | |
| LXR/RXR Activation | 3.67 | C4A/C4B, KNG1, APOE, AHSG, S100A8, APOC2, IL1F10, GC |
| Retinol Biosynthesis | 3.65 | CEL, LRAT, PNLIPRP1, PNPLA3, LIPG |
| Granulocyte Adhesion and Diapedesis | 3.16 | MMP3, MMP8, CXCL12, MMP10, MMP13, CXCL17, CLDN9, IL1F10, CLDN6 |
| Triacylglycerol Degradation | 3.13 | CEL, PNLIPRP1, PNPLA3, LIPG, PLA1A |
| SPINK1 Pancreatic Cancer Pathway | 2.92 | PRSS2, CPA1, CPZ, CTRB2, PRSS3 |
| Histamine Degradation | 2.72 | ALDH1A2, Aldh3b2, AOC1 |
| Leukocyte Extravasation Signaling | 2.65 | MMP3, MMP8, CXCL12, PIK3C2G, MMP10, MMP13, CLDN9, ACTG2, CLDN6 |
| Glutathione Redox Reactions I | 2.42 | GPX3, GPX2, GSTP1 |
| Role of Macrophages, Fibroblasts | 2.39 | SFRP2, WIF1, MMP3, PRSS2, CXCL12, PIK3C2G, MMP13, IL1F10, PDGFD, |
| and Endothelial Cells in Rheumatoid | | PRSS3, IL18R1 |
| Arthritis | | |
| Apelin Adipocyte Signaling Pathway | 2.37 | GPX3, GPX2, INS, Ins1, GSTP1 |
| Role of Osteoblasts, Osteoclasts and | 2.37 | COL1A1, SFRP2, WIF1, MMP3, MMP8, PIK3C2G, MMP13, IL1F10, IL18R1 |
| Chondrocytes in Rheumatoid Arthritis | | |
| Glycolysis I | 2.32 | ENO1, PKLR, ENO2 |
| Clathrin-mediated Endocytosis Signaling | 2.16 | APOE, SH3GL3, INS, PIK3C2G, S100A8, APOC2, ACTG2, PDGFD |
| Wnt/β-catenin Signaling | 2.09 | SOX17, GJA1, SFRP2, WIF1, NR5A2, SOX11, SOX5 |
| Acute Phase Response Signaling | 2.03 | C4A/C4B, HP, C1S, AHSG, IL1F10, A2M, C2 |
| LPS/IL-1 Mediated Inhibition of RXR | 2.00 | CHST2, APOE, ALDH1A2, HS6ST2, NR5A2, APOC2, IL1F10, |
| Function | | GSTP1 |
| PPAR Signaling | 1.95 | INS, PDGFRA, IL1F10, PDGFD, Ins1 |
| Role of IL-17A in Psoriasis | 1.91 | S100A9, S100A8 |
| Leukotriene Biosynthesis | 1.91 | DPEP1, GGT5 |
| Complement System | 1.89 | C4A/C4B, C1S, C2 |
| Asparagine Biosynthesis I | 1.88 | ASNS |
| Gap Junction Signaling | 1.67 | DBN1, GJA1, NOV, CAV1, GUCY1A2, PIK3C2G, ACTG2 |
| Graft-versus-Host Disease Signaling | 1.59 | IL1F10, HLA-E, HLA-DRB5 |
| HIF1α Signaling | 1.58 | MMP3, MMP8, PIK3C2G, MMP10, MMP13 |
| Chondroitin Sulfate Biosynthesis | 1.57 | CHST2, CHSY3, HS6ST2 |
| (Late Stages) | | |
| Oxidative Ethanol Degradation III | 1.51 | ALDH1A2, Aldh3b2 |
| OX40 Signaling Pathway | 1.50 | TNFSF4, H2-T22, HLA-E, HLA-DRB5 |
| Fatty Acid α-oxidation | 1.47 | ALDH1A2, Aldh3b2 |
| Actin Cytoskeleton Signaling | 1.46 | KNG1, MYH14, INS, PIK3C2G, ACTG2, PDGFD, Ins1 |
| Sertoli Cell-Sertoli Cell Junction | 1.45 | TJP3, GUCY1A2, CLDN9, ACTG2, CLDN6, A2M |
| Signaling | | |
| Bladder Cancer Signaling | 1.44 | MMP3, MMP8, MMP10, MMP13 |
| Putrescine Degradation III | 1.43 | ALDH1A2, Aldh3b2 |
| NF-κB Signaling | 1.41 | SIGIRR, INS, PDGFRA, PIK3C2G, IL1F10, Ins1 |
| Chondroitin Sulfate Biosynthesis | 1.40 | CHST2, CHSY3, HS6ST2 |
| PDGF Signaling | 1.38 | PDGFRA, CAV1, PIK3C2G, PDGFD |
| Tryptophan Degradation X (Mammalian, | 1.37 | ALDH1A2, Aldh3b2 |
| via Tryptamine) | | |
| Ethanol Degradation IV | 1.37 | ALDH1A2, Aldh3b2 |
| Dermatan Sulfate Biosynthesis | 1.36 | CHST2, CHSY3, HS6ST2 |
| Apelin Pancreas Signaling Pathway | 1.36 | INS, PIK3C2G, Ins1 |
| TR/RXR Activation | 1.36 | ENO1, HP, COL6A3, PIK3C2G |
| Gluconeogenesis I | 1.34 | ENO1, ENO2 |

TABLE 4
RNAseq - differentially expressed genes in KPC/CDH11^{+/-} versus KPC/CDH11^{+/+} [gemcitabine]

| | −log(p- | |
|---|---|---|
| Ingenuity Canonical Pathways | value) | Molecules |
| Autoimmune Thyroid Disease Signaling | 9.71 | HLA-DMA, HLA-DMB, HLA-DQA1, CD86, HLA-DQB1, IGHG1, Ighg2b, |
| | | HLA-E, HLA-DRB5 |
| B Cell Development | 9.39 | PTPRC, HLA-DMA, HLA-DMB, HLA-DQA1, CD86, HLA-DQB1, Ighg2b, |
| | | HLA-DRB5 |
| Antigen Presentation Pathway | 9.19 | HLA-DMA, HLA-DMB, HLA-DQA1, CIITA, HLA-DQB1, CD74, HLA-DRB5, |
| | | HLA-E |
| Allograft Rejection Signaling | 8.69 | H2-T22, HLA-DMA, HLA-DMB, HLA-DQA1, CD86, HLA-DQB1, IGHG1, |
| | | Ighg2b, HLA-E, HLA-DRB5 |
| Granulocyte Adhesion and Diapedesis | 7.54 | MMP7, SELL, MMP3, MMP15, CXCL14, FPR2, CCL21, CLDN9, CLDN6, |
| | | CCL19, SELPLG, ITGA4 |
| iCOS-iCOSL Signaling in T Helper Cells | 7.11 | PTPRC, IL2RG, HLA-DMA, HLA-DMB, ZAP70, HLA-DQA1, CD86, PIK3CD, |
| | | HLA-DQB1, HLA-DRB5 |
| Graft-versus-Host Disease Signaling | 6.94 | HLA-DMA, HLA-DMB, HLA-DQA1, CD86, HLA-DQB1, HLA-E, HLA-DRB5 |
| T Helper Cell Differentiation | 6.86 | IL2RG, HLA-DMA, HLA-DMB, HLA-DQA1, CD86, HLA-DQB1, GATA3, |
| | | HLA-DRB5 |
| Primary Immunodeficiency Signaling | 6.81 | PTPRC, IL2RG, Igha, ZAP70, CIITA, IGHG1, Ighg2b |
| Th2 Pathway | 6.32 | IL2RG, HLA-DMA, IKZF1, HLA-DMB, HLA-DQA1, CD86, PIK3CD, |
| | | HLA-DQB1, GATA3, HLA-DRB5 |
| Agranulocyte Adhesion and Diapedesis | 6.29 | MMP7, SELL, MMP3, MMP15, CXCL14, CCL21, CLDN9, CLDN6, CCL19, |
| | | SELPLG, ITGA4 |
| Dendritic Cell Maturation | 6.20 | HLA-DMA, HLA-DMB, CREB3L3, HLA-DQA1, CD86, PIK3CD, HLA- |
| | | DQB1, IGHG1, Ighg2b, FCGR1A, HLA-DRB5 |
| CD28 Signaling in T Helper Cells | 5.81 | PTPRC, HLA-DMA, HLA-DMB, ZAP70, HLA-DQA1, CD86, PIK3CD, |
| | | HLA-DQB1, HLA-DRB5 |
| Th1 and Th2 Activation Pathway | 5.50 | IL2RG, HLA-DMA, IKZF1, HLA-DMB, HLA-DQA1, CD86, PIK3CD, |
| | | HLA-DQB1, GATA3, HLA-DRB5 |
| Nur77 Signaling in T Lymphocytes | 5.08 | HLA-DMA, HLA-DMB, HLA-DQA1, CD86, HLA-DQB1, HLA-DRB5 |
| Altered T Cell and B Cell Signaling | 5.06 | HLA-DMA, HLA-DMB, HLA-DQA1, CCL21, CD86, HLA-DQB1, HLA-DRB5 |
| in Rheumatoid Arthritis | | |
| OX40 Signaling Pathway | 5.06 | H2-T22, HLA-DMA, HLA-DMB, HLA-DQA1, HLA-DQB1, HLA-E, HLA-DRB5 |
| Cdc42 Signaling | 5.03 | H2-T22, PAK3, HLA-DMA, HLA-DMB, HLA-DQA1, HLA-DQB1, |
| | | HLA-E, HLA-DRB5, ITGA4 |
| PKCθ Signaling in T Lymphocytes | 5.03 | RAC2, HLA-DMA, HLA-DMB, ZAP70, HLA-DQA1, CD86, PIK3CD, HLA- |
| | | DQB1, HLA-DRB5 |
| Leukocyte Extravasation Signaling | 5.00 | RAC2, MMP7, MMP3, MMP15, PIK3CD, CLDN9, CLDN6, RHOH, SELPLG, |
| | | ITGA4 |
| IL-4 Signaling | 4.87 | IL2RG, HLA-DMA, HLA-DMB, HLA-DQA1, PIK3CD, HLA-DQB1, HLA-DRB5 |
| Calcium-induced T Lymphocyte Apoptosis | 4.80 | HLA-DMA, ZAP70, HLA-DMB, HLA-DQA1, HLA-DQB1, HLA-DRB5 |
| Th1 Pathway | 4.78 | HLA-DMA, HLA-DMB, HLA-DQA1, CD86, PIK3CD, HLA-DQB1, GATA3, |
| | | HLA-DRB5 |
| T Cell Exhaustion Signaling Pathway | 4.73 | HLA-DMA, HLA-DMB, ZAP70, HLA-DQA1, CD86, PIK3CD, HLA-DQB1, |
| | | HLA-E, HLA-DRB5 |
| Role of NFAT in Regulation of the | 4.53 | HLA-DMA, HLA-DMB, ZAP70, HLA-DQA1, CD86, PIK3CD, HLA-DQB1, |
| Immune Response | | FCGR1A, HLA-DRB5 |
| Type I Diabetes Mellitus Signaling | 4.46 | HLA-DMA, HLA-DMB, HLA-DQA1, CD86, HLA-DQB1, HLA-E, HLA-DRB5 |
| Neuroinflammation Signaling Pathway | 4.27 | MMP3, KLK3, HLA-DMA, HLA-DMB, CREB3L3, HLA-DQA1, CD86, |
| | | PIK3CD, HLA-DQB1, PTGS2, HLA-DRB5 |
| Phagosome Formation | 3.98 | CLEC7A, PIK3CD, IGHG1, Ighg2b, FCGR1A, RHOH, ITGA4 |
| IL-7 Signaling Pathway | 3.95 | IL2RG, Igha, PIK3CD, IGHG1, Ighg2b, Ighg2c |
| Communication between Innate and Adaptive | 3.87 | Igha, CD86, IGHG1, Ighg2b, HLA-E, HLA-DRB5 |
| Immune Cells | | |
| Systemic Lupus Erythematosus Signaling | 3.85 | PTPRC, SNRPN, CD22, CD86, PIK3CD, IGHG1, Ighg2b, FCGR1A, HLA-E |
| B Cell Receptor Signaling | 3.71 | PTPRC, RAC2, CREB3L3, CD22, PIK3CD, IGHG1, BCL2A1, Ighg2b |
| ErbB Signaling | 2.76 | BTC, PAK3, PIK3CD, EREG, AREG |
| Prostanoid Biosynthesis | 2.64 | PTGS2, PTGDS |
| Phospholipase C Signaling | 2.41 | PLD4, CREB3L3, ZAP70, IGHG1, Ighg2b, RHOH, ITGA4 |
| Inhibition of Matrix Metalloproteases | 2.41 | MMP7, MMP3, MMP15 |
| Regulation of Actin-based Motility by Rho | 2.22 | RAC2, PAK3, RHOH, ITGA4 |
| Crosstalk between Dendritic Cells and | 2.22 | IL2RG, CD86, HLA-DRB5, HLA-E |
| Natural Killer Cells | | |
| Role of Oct4 in Mammalian Embryonic Stem | 2.21 | CDX2, IGF2BP1, SALL4 |
| Cell Pluripotency | | |
| Fcγ Receptor-mediated Phagocytosis in | 2.17 | PLD4, RAC2, FCGR1A, FGR |
| Macrophages and Monocytes | | |
| Neuregulin Signaling | 2.15 | BTC, EREG, AREG, ITGA4 |
| Hematopoiesis from Pluripotent Stem Cells | 2.13 | Igha, IGHG1, Ighg2b |
| Tec Kinase Signaling | 1.89 | PAK3, PIK3CD, RHOH, FGR, ITGA4 |
| Fatty Acid α-oxidation | 1.87 | ALDH1A3, PTGS2 |
| Germ Cell-Sertoli Cell Junction Signaling | 1.80 | RAC2, PAK3, LAMC3, PIK3CD, RHOH |
| Role of Macrophages, Fibroblasts and | 1.78 | WIF1, MMP3, CREB3L3, PIK3CD, IGHG1, Ighg2b, FCGR1A |
| Endothelial Cells in Rheumatoid Arthritis | | |
| Neuroprotective Role of THOP1 in | 1.78 | KLK3, PRSS12, CTRB2, HLA-E |
| Alzheimer's Disease | | |
| Gαi Signaling | 1.75 | OPRD1, P2RY13, FPR2, P2RY12 |
| Eicosanoid Signaling | 1.74 | FPR2, PTGS2, PTGDS |
| Colorectal Cancer Metastasis Signaling | 1.74 | MMP7, MMP3, MMP15, PIK3CD, PTGS2, RHOH |
| HIF1α Signaling | 1.71 | MMP7, MMP3, MMP15, PIK3CD |
| Atherosclerosis Signaling | 1.69 | MMP3, CCR2, ITGA4, SELPLG |
| PI3K/AKT Signaling | 1.67 | GDF15, PIK3CD, PTGS2, ITGA4 |
| Natural Killer Cell Signaling | 1.66 | RAC2, PAK3, ZAP70, PIK3CD |
| MSP-RON Signaling Pathway | 1.65 | KLK3, PIK3CD, CCR2 |
| TREM1 Signaling | 1.63 | TREM1, CIITA, CD86 |
| Agrin Interactions at Neuromuscular Junction | 1.63 | RAC2, PAK3, ITGA4 |
| VDR/RXR Activation | 1.59 | SERPINB1, HR, HSD17B2 |
| IL-8 Signaling | 1.59 | PLD4, RAC2, PIK3CD, PTGS2, RHOH |
| ERK/MAPK Signaling | 1.58 | RAC2, PAK3, CREB3L3, PIK3CD, ITGA4 |
| G-Protein Coupled Receptor Signaling | 1.55 | OPRD1, P2RY13, CREB3L3, FPR2, P2RY12, PIK3CD |
| MIF-mediated Glucocorticoid Regulation | 1.51 | CD74, PTGS2 |
| Retinoate Biosynthesis I | 1.51 | ALDH1A3, RBP2 |
| Neurotrophin/TRK Signaling | 1.51 | KLK3, CREB3L3, PIK3CD |
| Retinoate Biosynthesis II | 1.49 | RBP2 |
| Estrogen-Dependent Breast Cancer Signaling | 1.47 | CREB3L3, PIK3CD, HSD17B2 |
| Integrin Signaling | 1.46 | RAC2, PAK3, PIK3CD, RHOH, ITGA4 |
| Dopamine Degradation | 1.44 | ALDH1A3, Sult1a1 |
| cAMP-mediated signaling | 1.42 | OPRD1, P2RY13, CREB3L3, FPR2, P2RY12 |
| Creatine-phosphate Biosynthesis | 1.40 | CKMT1A/CKMT1B |
| IL-17 Signaling | 1.40 | MMP3, PIK3CD, PTGS2 |
| Reelin Signaling in Neurons | 1.38 | PIK3CD, FGR, ITGA4 |
| Bladder Cancer Signaling | 1.38 | MMP7, MMP3, MMP15 |
| Estrogen Biosynthesis | 1.36 | HSD17B2, CYP2S1 |
| MIF Regulation of Innate Immunity | 1.34 | CD74, PTGS2 |
| CTLA4 Signaling in Cytotoxic T Lymphocytes | 1.31 | ZAP70, CD86, PIK3CD |

CDH11-Targeting Combined with Gemcitabine or Anti-PD1

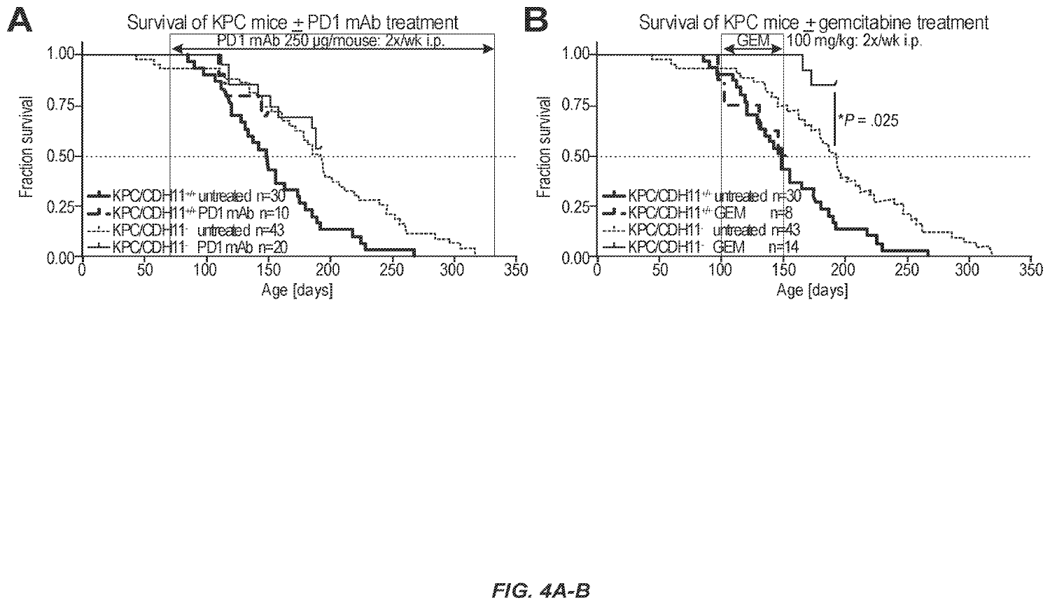

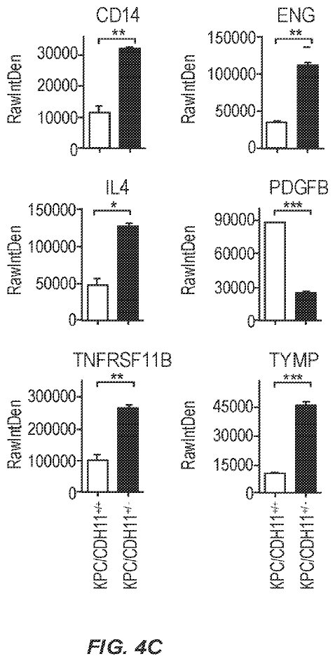

Even though the effects of CDH11-targeting alone on survival are substantial, whether the combination of CDH11-targeting with currently available immunotherapy (PD1 mAb), or commonly used chemotherapy in PDAC patients, gemcitabine, could further extend survival was assessed. Although KPC/CDH11^{+/+} mice showed a trend towards improved survival following treatment with anti-PD1, this was not statistically significant, and KPC/CDH11-deficient animals did not show any hint of further improvement after PD1 mAb therapy (FIG. 4A). While PD1 (PDCD1) expression in KPC/CDH11^{+/-} mice increased 1.8-fold, a slight reduction (−1.3-fold) in PDL1 (CD274) was detected. Interferon 7, a major regulator of PDL1 levels in other systems, was also unaffected by CDH11 deficiency. Hence, PDAC tumors in KPC/CDH11-deficient animals may depend on non-PD1/PDL1 pathways, such as alternative inhibitory checkpoints.

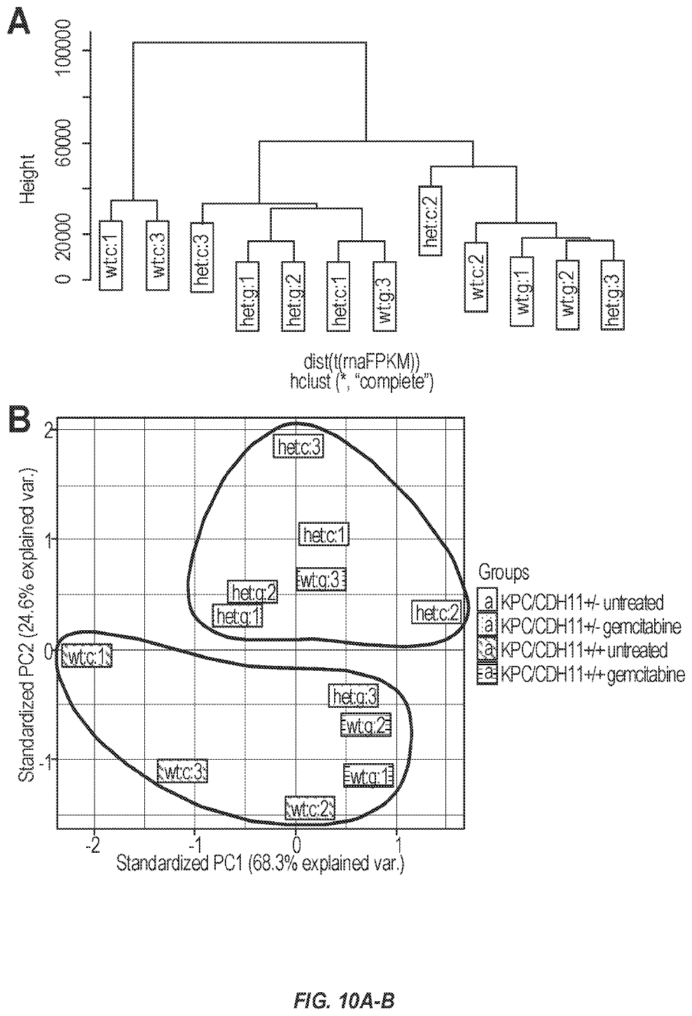

Gemcitabine was used as a nucleoside analog that inhibits DNA synthesis upon incorporation into DNA. Starting at 100 days of age, KPC mice were treated for 6 weeks, equivalent to a single course of chemotherapy in the human disease. Remarkably, the first PDAC-related death in the KPC/CDH11-deficient group occurred only after gemcitabine treatment ended (at 165 days), much later than in the KPC/CDH11^{+/+} mice, and its overall survival improved significantly over the KPC/CDH11-deficient untreated group (FIG. 4B). However, hierarchical clustering and principal component analysis of RNAseq data suggests that genotype (CDH11 status) has a larger impact on the transcriptome than gemcitabine treatment (FIG. 10). RNAseq analysis revealed a marked increase in the expression of numerous immunoglobulins and antigen presentation genes (H2-Eb1, H2-T22, H2-T23, Ciita, H2-DMb1, H2-Abl, Cd74, H2-Aa, H2-DMa, H2-K1) in PDAC tissues from gemcitabine-treated KPC/CDH11^{+/-} mice compared to gemcitabine-treated KPC/CDH11^{+/+} animals (Table 4). In addition, CCL21 (attracts T and dendritic cells) and CXCL13 (attracts B cells) were both increased >5 and >2-fold, respectively (FIG. 4D, FIG. 9C, Table 2). IL4, which activates B cells, increased >2-fold in serum of KPC/CDH11^{+/-} gemcitabine-treated mice (FIG. 4C, FIG. 9B, Table 2). Together, these data suggest increased PDAC infiltration with B cells in KPC/CDH11^{+/-} animals upon gemcitabine treatment. Ingenuity Pathway Analysis also indicated changes in B cell development, antigen presentation, and T cell differentiation, signaling and activation pathways, and dendritic cell maturation (Table 4). Finally, protein levels of multiple other cytokines associated with tumor progression or poor prognosis (e.g. IL11, VEGFA, PTX3, CRP and IL1A)⁴⁰⁻⁴³ were decreased in tumor tissues of gemcitabine-treated KPC/CDH1^{+/-} mice in comparison to gemcitabine-treated KPC/CDH11^{+/+} mice (FIG. 4D, FIG. 9C, Table 2).

CDH11 mAb in Combination with Gemcitabine in mT3 Transplanted Mice

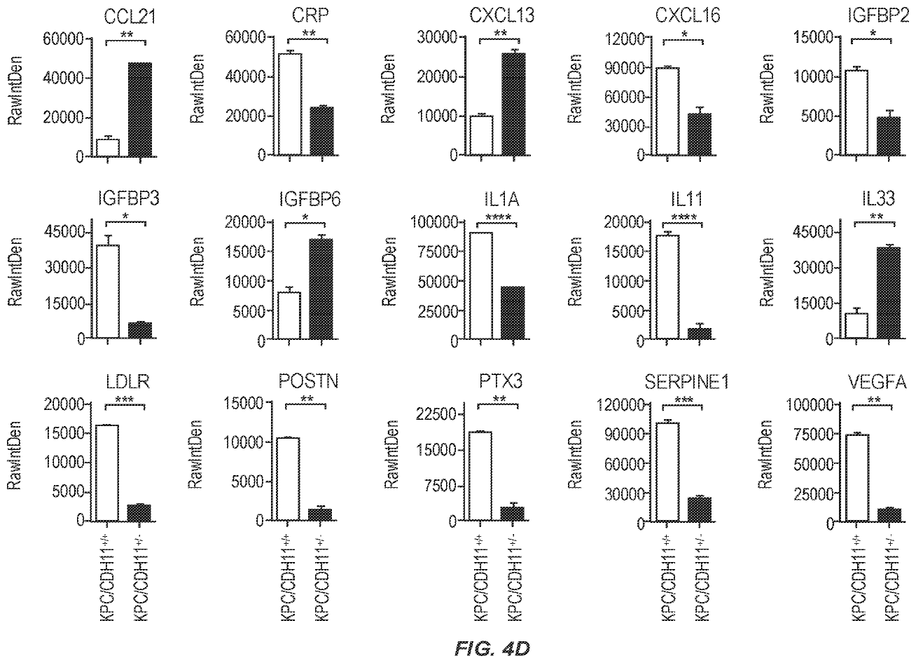

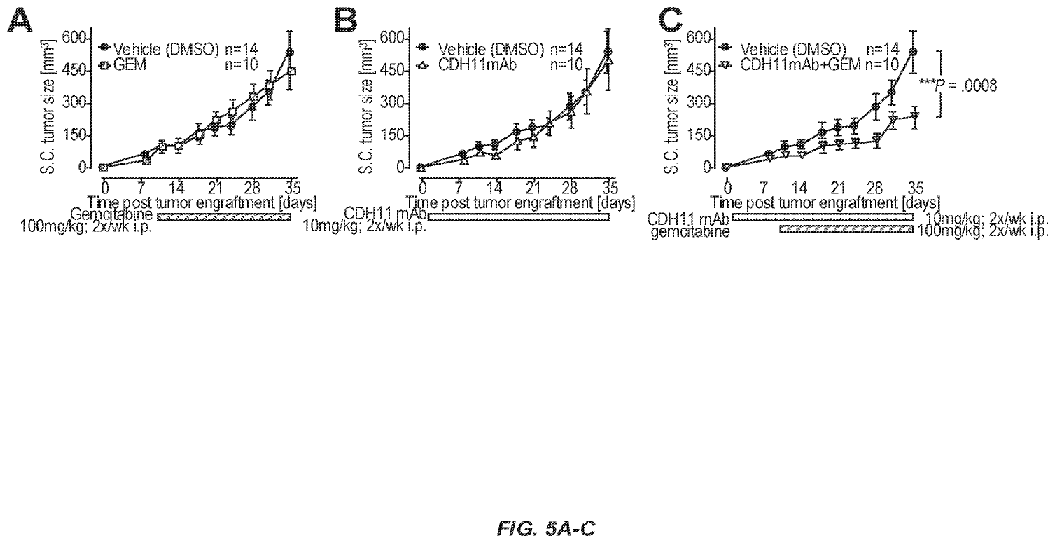

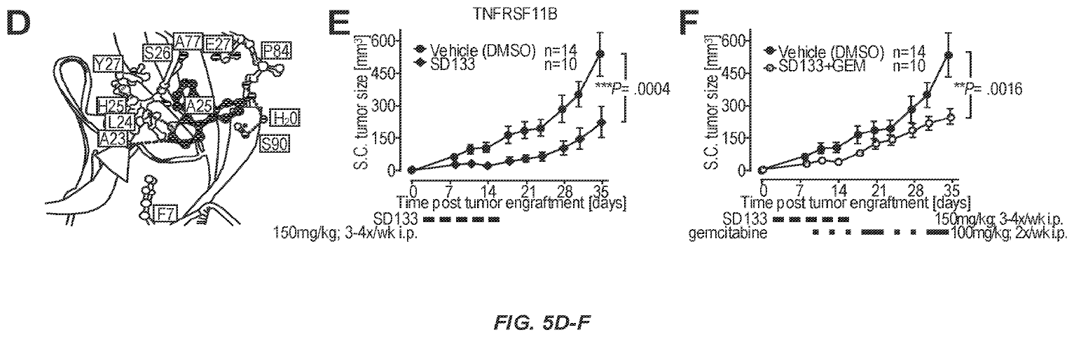

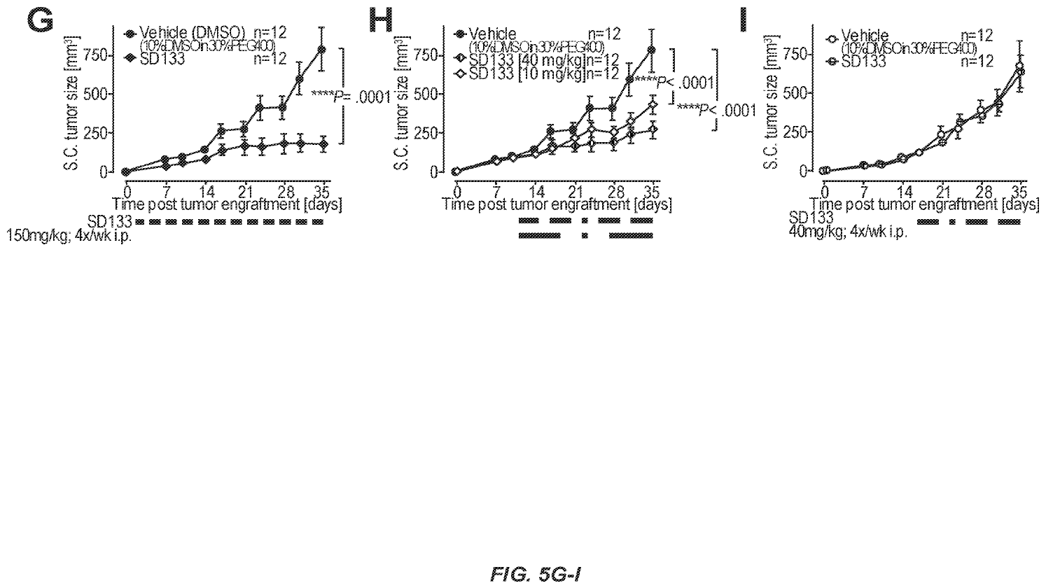

Next, a CDH11 mAb, which targets the cell-to-cell adhesion-binding region of CDH11, was used in an immunocompetent transplant mouse model of pancreatic cancer. C57BL/6J mice were s.c. injected with mT3 pancreatic cancer cells, derived from a Kras^{+/LSL-G12D};Trp53^{+/LSL-R172H};Pdx-Cre mouse on C57BL/6 background. Engrafted mice were treated with a CDH11 mAb (SYN0012)(Chang et al. J. Clin. Invest. 127: 3300-3312 (2017)), with or without addition of gemcitabine. As expected, gemcitabine-only treatment did not reduce tumor growth (FIG. 5A). Although treatment with CDH11 mAb alone also failed to reduce tumor growth (FIG. 5B), combination of CDH11 mAb plus gemcitabine reduced in tumor growth (FIG. 5C). The combinatorial effect observed here is somewhat similar to the outcome in the gemcitabine-treated KPC/CDH11^{+/-} mice.

Treatment with Small Molecule CDH11-Inhibitor Reduces Pancreatic Tumor Growth

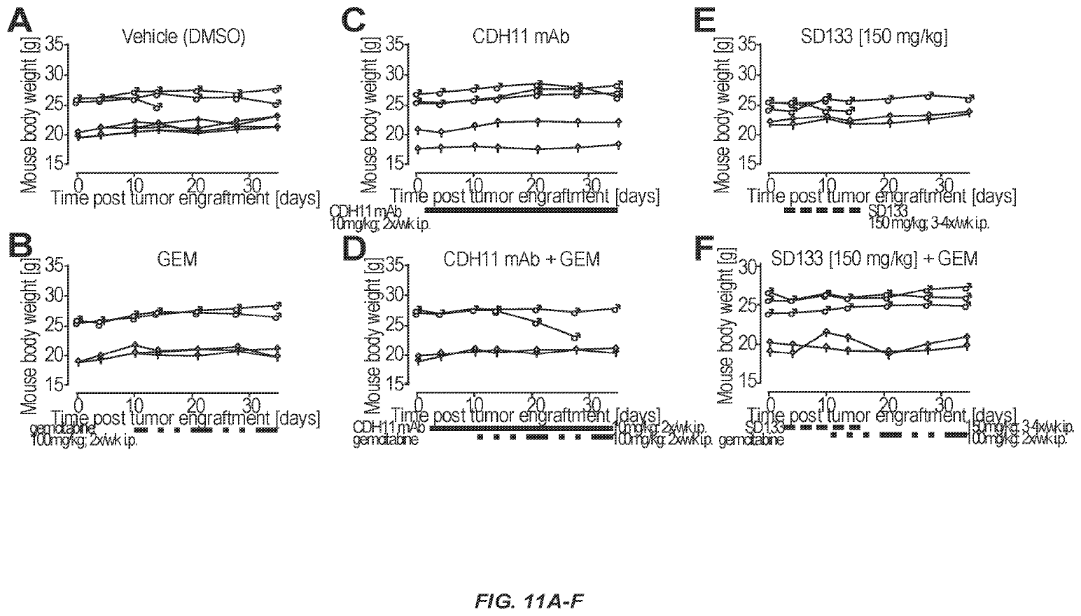

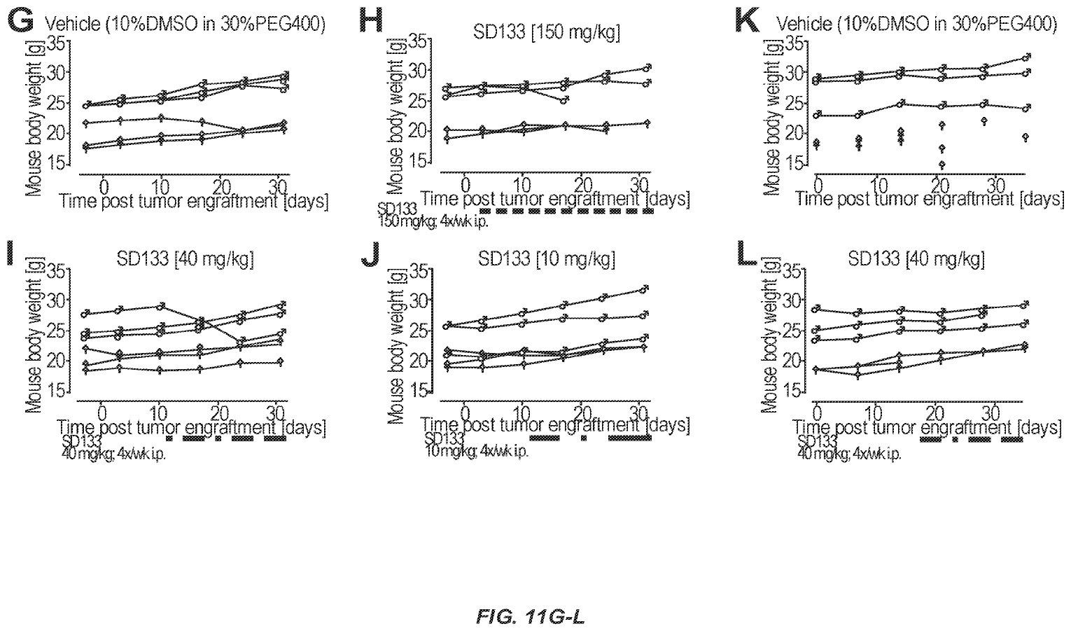

Previously, several families of drug-like small molecule inhibitors of CDH11 were designed. One of these, SD133, binds specifically to the CDH11 binding pocket (FIG. 5D) with low μM affinity in vitro, and it was previously characterized and validated. mT3 tumor-bearing C57BL/6J mice were treated with SD133 beginning on day 2, after cancer cell injection, and d a marked reduction in tumor growth (FIG. 5E) was observed, with no decrease in animal weight during the experiment (FIG. 11). As SD133 treatment effectively attenuated tumor growth during the administration period, a combinatorial effect with gemcitabine (FIG. 5F) was not observed. After the treatment with SD133 discontinued, tumors began to grow, but they remained significantly smaller than tumors in control animals. In the second experiment, treatment was continued throughout the study and reduced tumor growth was observed for the duration of the treatment (FIG. 5G). In a separate experiment, we started treatment once the average tumor volume reached 100 mm³. SD133 treatment at 40 mg/kg and 10 mg/kg significantly reduced growth rate of pre-existing tumors in a dose-dependent manner (FIG. 5H). Collectively, our data confirm that therapeutic inhibition of CDH11, perhaps in combination with gemcitabine, constrains the progression of PDAC tumors.

### Effective Attenuation of Pancreatic Tumor Growth Upon CDH11-Targeting Requires the Presence of T and B Cells

Finally, to further explore the immunomodulatory mechanism of CDH11-inhibition in the pancreatic cancer context, immunocompromised Rag1-mutant mice (on C57BL/6J background) were engrafted with mT3 cells, derived from PDAC of a C57BL/6 mouse. After s.c. tumors reached an average volume of 100 mm³, treatment was started with 40 mg/kg of SD133, the dose that was effective in immunocompetent C57BL/6J mice. Interestingly, in the absence of mature T and B cells, the small molecule CDH11-inhibitor, SD133 did not suppress tumor growth indicating that T and B cells are necessary components of CDH11-mediated immunomodulation of the PDAC TME (FIG. 5I).

As shown herein, CDH11 is primarily expressed by CAFs in stroma of human and mouse PDAC. CDH11-deficiency correlates with: (1) decreased stromal activation, and collagen and fibronectin expression in PDAC, consistent with the demonstration that fibroblasts isolated from CDH11^{+/+} mice produce more collagen than those from CDH11-deficient mice. Moreover, reduced αSMA expression around early PanIN lesions in KPC/CDH11-deficient mice suggests a delayed stromal activation, which may ultimately translate into sustained or delayed tumor progression. Importantly, a battery of approaches, including RNAseq, Nanostring, cytokine and phosphoprotein arrays, showed that CDH11-deficiency contributes to anti-tumor immunity by modulating the PDAC TME through: (2) decreasing markers associated with immunosuppressive cells, such as MDSCs and M2 macrophages; and (3) promoting expression of antigen processing and presentation genes as well as professional antigen presenting cell markers, such as dendritic cells, M1 macrophages and B cells. Both outcomes are likely mediated by secreted cytokines that attract or deter specific immune cells, and/or skew immune cell differentiation.

For example, CDH11-deficiency leads to a reduced number of FOXP3⁺ cells in the tumor center—possibly driven by decreased expression of CCL17 and CCL22; however, it does not cause complete T-reg depletion associated with myeloid cell infiltration, even in KPC/CDH11-mice. Expression of myeloid-recruiting cytokines Ccl3, Ccl6 and their receptor Ccr1 decreased in pancreata of KPC/CDH11^{+/-} mice in comparison to KPC/CDH11^{+/+}. Additionally, decreased MDSC-associated markers and reduced fibrosis could be partially attributed to a decreased IL11 expression in KPC/CDH11^{+/-} mice, since IL11 plays a role in promoting pulmonary fibrosis and inducing differentiation of MDSCs.

Even though correlative, the data provided herein suggests that localization of FOXP3⁺ cells plays a role in mitigating tumor growth. Specifically, the reduced number of T-regs in the tumor center of KPC/CDH11-deficient mice correlates with a decreased fibroblast activation, increased expression of genes associated with antigen processing and presentation, increased signaling through PKC-θ and SLP-76 suggesting greater T cell activation downstream of the TCR complex and ultimately longer survival.

In the models described herein, CDH11 is depleted or inhibited in the whole animal, rather than specifically in a particular cell type. However, in post-embryonic life, CDH11 expression is largely restricted to activated fibroblasts, osteosarcomas and some invasive carcinomas. In PDAC, CDH11 is almost exclusively expressed by CAFs, and consequently, the data provided herein suggest that the effects of CDH11 deficiency or inhibition are mediated by CAFs. CDH11 protein may also be present in particular macrophage sub-types in other diseases, but we did not observe this in PDAC (FIG. 1H). Since CDH11 could be cleaved and released from the cell surface similarly to E-cadherin, and it could activate growth factor receptors on other cells, CDH11 could potentially be taken up by macrophages, perhaps explaining the presence of protein but not RNA in these cells in other systems. As both the antibody and small molecule inhibitors target the cell-to-cell adhesion pocket, it is likely that this region mediates both autonomous and non-autonomous actions.

In summary, CDH11 is an excellent target for PDAC treatment, because it is not widely expressed in non-diseased tissues in post-embryonic life, but is highly expressed on activated fibroblasts in the stroma of PDAC patients. A marked survival advantage observed across several immunocompetent PDAC mouse models suggests that targeting CDH11 has a great potential for clinical translation. Even a 2.45-fold reduction in CDH11 expression in KPC/CDH11^{+/+} animals, significantly increased survival, suggesting that complete disruption of CDH11 function is unnecessary, which bodes well for the development of CDH11-targeted therapy. The drug-like small molecule CDH11-inhibitor, SD133, was remarkably effective as a monotherapy, even on pre-existing tumors, with no sign of toxicity. Conversely, a CDH11 mAb in clinical trials for other indications was less effective as a monotherapy but yielded a significant combinatorial effect with gemcitabine and could feasibly be translated into the clinic.

1. A composition comprising, in a solution:
a) about 5% to about 15% of an organic solvent (w/w);
b) about 20% to about 45% polyethylene glycol (PEG) (w/w); and
c) a compound of the following formula: 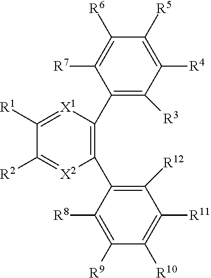
or a pharmaceutically acceptable salt or prodrug thereof, wherein:
R¹, R², R³, R⁴, R⁵, R⁶, R⁷, R⁸, R⁹, R¹⁰, R¹¹, and R¹² are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and
X¹ and X² are each independently selected from CH or N.

2. The composition of claim 1, wherein the organic solvent is dimethyl sulfoxide (DMSO).

3. The composition of claim 1, wherein the PEG is selected from the group consisting of PEG-400, PEG-100, PEG-200, PEG-300, PEG-600, PEG-800, PEG-1000, PEG-2000, PEG-3000, PEG-4000, PEG-5000, PEG-6000, PEG-7000, PEG-8000, PEG-9000, and PEG-10,000.

4. The composition of claim 1, wherein the compositions comprises about 10% DMSO and about 30% PEG-400.

5. The composition of claim 1, wherein the compound has the following formula 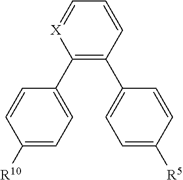
or a pharmaceutically acceptable salt or prodrug thereof, wherein:
R⁵ and R¹⁰ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl; and
X is selected from CH or N.

6. The composition of claim 4, wherein the compound is 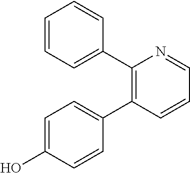 [SMILES: Oc1ccc(-c2cccnc2-c2ccccc2)cc1]
or a pharmaceutically acceptable salt or prodrug thereof.

7. The composition of claim 1, wherein the compound has the following formula: 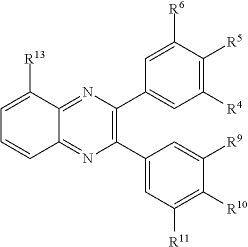
or a pharmaceutically acceptable salt or prodrug thereof, wherein:
R⁴, R⁵, R⁶, R⁹, R¹⁰, R¹¹, and R¹³ are each independently selected from hydrogen, halogen, hydroxyl, substituted or unsubstituted alkoxy, substituted or unsubstituted amido, substituted or unsubstituted amino, substituted or unsubstituted carbonyl, substituted or unsubstituted alkyl, unsubstituted or unsubstituted alkenyl, substituted or unsubstituted alkynyl, unsubstituted heteroalkyl, unsubstituted or unsubstituted heteroalkenyl, substituted or unsubstituted heteroalkynyl, substituted or unsubstituted cycloalkyl, unsubstituted or unsubstituted cycloalkenyl, substituted or unsubstituted cycloalkynyl, substituted or unsubstituted heterocycloalkyl, unsubstituted or unsubstituted heterocycloalkenyl, substituted or unsubstituted heterocycloalkynyl, substituted or unsubstituted aryl, or substituted or unsubstituted heteroaryl.

8. The composition of claim 7, wherein the compound is: 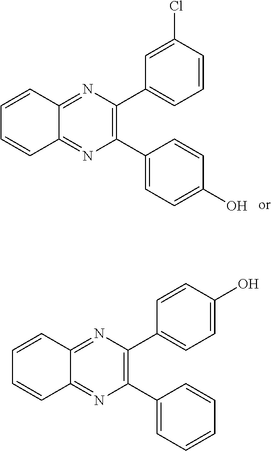 [SMILES: Oc1ccc(-c2nc3ccccc3nc2-c2cccc(Cl)c2)cc1 ; "or" ; Oc1ccc(-c2nc3ccccc3nc2-c2ccccc2)cc1]
or a pharmaceutically acceptable salt or prodrug thereof.

9. The composition of claim 1, further comprising a chemotherapeutic agent.

10. The composition of claim 1, further comprising an immunotherapeutic agent.

11. The composition of claim 10, wherein the immunotherapeutic agent is a checkpoint inhibitor.

12. The composition of claim 11, wherein the checkpoint inhibitor is selected from the group consisting of a BTLA inhibitor, a CTLA4 inhibitor and a LAG3 inhibitor.

13. A method for treating a cadherin-11 related disease in a subject comprising administering to the subject in need thereof an effective amount of:
a) the composition of claim 1; and
b) a chemotherapeutic agent or an immunotherapeutic agent.

14. The method of claim 13, wherein the cadherin-11 related disease is refractory to the chemotherapeutic agent or the immunotherapeutic agent.

15. The method of claim 13, wherein administration of the composition enhances the efficacy of the chemotherapeutic agent or the immunotherapeutic agent.

16. The method of claim 13, wherein the composition decreases fibrosis in the subject.

17. The method of claim 13, wherein the cadherin-11 related disease is cancer.

18. The method of claim 17, wherein the cancer is pancreatic cancer.

19. The method of claim 13, wherein the chemotherapeutic agent is gemcitabine.

20. The method of claim 13, wherein the immunotherapeutic agent is a checkpoint inhibitor.

21. The method claim 20, wherein the checkpoint inhibitor is selected from the group consisting of a BTLA inhibitor, a CTLA4 inhibitor and a LAG3 inhibitor.

22. The method of claim 13, wherein the cadherin-11 related disease is an autoimmune disorder.
